SEQUENCE LISTING (38 sequences)

SEQ ID NO: 1 - DNA, 2084 bp, Solanum tuberosum
  CDS (302)..(1696)
ttttcataaacttcttcaactttattccatactcttttatttatcagctcctagatcttcttttttgtttgttgatattctcttgaaaattgttcagtgaagagttgatcaaagctaagacacaggggctgcggccattttttcaccggaatcttcttctttattttccggtgaaagtttgaacgcacaccgttatttctagacagtagacaatgtcaagtgaaaaacatcacaagtttttgaagatttgtaattaattagttgagatttttaatttggaggaaagagaaaaacagagaagatgatagggcgggtgggcttgttgttggtattgttgatagcaacgacggtgactattggggctgaaacgacgacgttaaaaggggtaaacagaaatgcgtatgcgactatgatgtatatgggaactccgagagactacgagttctacgtggcgactcgagtaatgctccgatcacttacccggctaggagttgaagccgatctcgtcgttattgcttcacttgacgttcctcttcgctgggttcaaactctagaacaggaagatggtgctaaggtggtgagagttaaaaatctgaacaatccgtattgtatcaaccctaattggagattcaagctcacactgaacaaactttatgcgtggagcctcgtaaattatgacagggttgtcatgcttgatgctgacaaccttttcctccagaaaactgatgaactgttccaatgtggccagttttgtgctgtcttcattaatccctgcatcttccacactggtctctttgtattgcagccatcaaaaaaggtgttcaatgacatgatccatgagatagagattgggagggaaaatcaagacggtgcagaccaaggttttattggaggccacttcccagatttacttgatcggccaatgttccaccctcctcttaatggtacccagctccagggaagttacaggcttcctctaggataccaaatggacgcctcttattattatctcaaactccattggtcggtaccttgtggacctaatagtgtcattacatttcctggtgctccatggttaaaaccatggtattggtggtcatggcctgtcttacccttgggcatccagtggcatgaacagcgacgtctaactgttgggtatggtgctgagatgatagcagtgttgatccaatctatattttacctaggaataattgcagtgacacgcctagcacgcccaaatttatcaaagttgtgctatcgccatgatgatagcaagagtgccttcttactacgaactggccttaaattgattgctatatggtccattcttgctgcctacacagttccttatttcgtgattccttgtacagttcatccactagttggctggagtctctacttactcggctctttttcactatcctgtataacagtgaatgcatttcttttgccgatgctacctgttttagtcccatggattgggatccttggggcccttttggtgatggcttacccttggtacaacgacggtgttgtaagagcaatggctgtatttacatacgccttctgtgcttctccagcattatggatggcattggttaaaatcaagtgttctcttcatgtttcacttgagagggaaggattcttgcccaagataagtgaatctacagcacctgctggttctaacaaactgtattgaaagttgaaaagttaaaggaatcaacaggagaactaatgcttcagaaacatctccaaacgttttgcttaggagacttggagtctgcttgtgctatcctagctagttgcttcagtctgtgctcttaattagaatggaattctgtgagtgggtttagaattgggaggatgttttgtgttgtacatggactatctctggtctcttgaatgctactccaggaaaaagattgtttctcacttaattttttctgttactaaattgtatgtggaataggttctttaaaatttattcatggatttatgttatgtatgctaacagtgtaaatattaagtcctggtgaaataagtaattccttattcatacaaaaaaaaaaaaaaaaaaaaaaaaa

SEQ ID NO: 2 - protein, 465 aa, Solanum tuberosum
MIGRVGLLLVLLIATTVTIGAETTTLKGVNRNAYATMMYMGTPRDYEFYVATRVMLRSLTRLGVEADLVVIASLDVPLRWVQTLEQEDGAKVVRVKNLNNPYCINPNWRFKLTLNKLYAWSLVNYDRVVMLDADNLFLQKTDELFQCGQFCAVFINPCIFHTGLFVLQPSKKVFNDMIHEIEIGRENQDGADQGFIGGHFPDLLDRPMFHPPLNGTQLQGSYRLPLGYQMDASYYYLKLHWSVPCGPNSVITFPGAPWLKPWYWWSWPVLPLGIQWHEQRRLTVGYGAEMIAVLIQSIFYLGIIAVTRLARPNLSKLCYRHDDSKSAFLLRTGLKLIAIWSILAAYTVPYFVIPCTVHPLVGWSLYLLGSFSLSCITVNAFLLPMLPVLVPWIGILGALLVMAYPWYNDGVVRAMAVFTYAFCASPALWMALVKIKCSLHVSLEREGFLPKISESTAPAGSNKLY

SEQ ID NO: 3 - DNA, 2230 bp, Solanum tuberosum
  CDS (143)..(2086)
tatccccagagaatcagctgaatcaagaactgatttttagattatgttttcttgattctttgaaatgggaacttgattttcagtttttcaactcagatgttgtgttcctttagctggaaaacttgaaaaaggaaagcccagaatgagaggaagtttagctggtggaccacctagtcctattgaacctagacagaggctttctgtattcactgaggaaacaagcaaaagaaggttcttgagaagtaaagttttcagagatggggagagagctcttcatagtcccaccaaaaacaggaattttacctgcaagttcccaactgtgaagcttatattgggtgttattgctctggttgcaatttggtcactctggcattctccagcaatttataacacggaatacatatctagttcaggctctcgggctgctttgatgcacagagagttaagtggtcattcttcagctgatcaacgttatacatcacttttagatattgactgggaccaaatttcccaagttattgagaaactggccgataggcatgagtatcagggcgtagggatattaaacttcaatgacagtgaaattgatcagttgaaggagttactaccggacgctgagcatgtaatcttgaacctggatcacgtcccgaataatataacatgggaaacaatatatcctgaatggatagatgaagaagaagaatttgaggtccccacttgtccttctctgcccaaaattcagtttccgggtaaaccaaggattgatctcatagttgtaaagcttccatgcaagaagtctaaggactggtatagagatgtagctcgttttcacttgcagctggcagcagcaaggctggctgccagtaataaagggtatcatccaatacatgtgcttctggttactgagcatttcccaacccccaatctgttcacctgtaaagagttagttgtacgtgaaggcaatgcatggctatatgaacctaatctgaacactttaagagagaagctccacctccctgttgggtcatgtgaacttgcagttcctctcaaggctaaagcaaattggcactctggaaatgtaagacgagaagcctatgcaactattctccactcagcaaatttttatgtatgtggagccatagctgcagcacagagtattcgcttggcaggttcaacccgagatcttgtgatacttgttgatgagactatcagtgactaccacaggggtggtttagaggctgccggatggaagatccacacgataaagagaataaggaatcctaaagctgaacaggatgcctacaatgagtggaactatagcaaatttcgtctctggcagctgacagattatgacaaaatcatcttcattgatgcggatttgttgatactgagaaatattgattttctctttgagatgcctgaaataactgcaataggaaataatgcaaccctttttaattcaggcgtgatggtcgttgaaccatcaaattgcacatttcagctgttgatggatcatatcaatgagattgaatcatacaatggtggggatcaggggtatttgaacgaaatattcacctggtggcataggatcccaaaacacatgaactttttgaaacattattgggaaggtgatgaggaggagaagaagcaaatgaaaacacgtctttttggtgctgatcctccagttctctatgttctgcactacttaggcctgaaaccttggttatgcttcagggactacgattgcaactggaatgtgggtaagttgcaggagtttgcaagtgatgtggcacacaggacgtggtggaaggtccatgatgccatgccagataacttacataaatattgtttgcttaggtctaaacagaaggctgcactagagtgggatcgaagagaagctgagaaagctaacttttcagatggtcattggaagatcaaaataaaggacccacgtttggagacttgttatgaagaattttgcttctgggaaagcatgttatggcactggggtgaaacaaactggacagataatgccacctcttcaccaacacctcccatggtcaatactgcttcactttcttctttgtaactaggaggttctgttactatacctcgctagtctgtaagtaatacagagtcacaacttgaatgtcaaccagtgccattagtatacatggtgtcaacactttccccacccttgaaaaaaaaaaaaaaaaaaaaaaaaaaaaaa

SEQ ID NO: 4 - protein, 648 aa, Solanum tuberosum
MRGSLAGGPPSPIEPRQRLSVFTEETSKRRFLRSKVFRDGERALHSPTKNRNFTCKFPTVKLILGVIALVAIWSLWHSPAIYNTEYISSSGSRAALMHRELSGHSSADQRYTSLLDIDWDQISQVIEKLADRHEYQGVGILNFNDSEIDQLKELLPDAEHVILNLDHVPNNITWETIYPEWIDEEEEFEVPTCPSLPKIQFPGKPRIDLIVVKLPCKKSKDWYRDVARFHLQLAAARLAASNKGYHPIHVLLVTEHFPTPNLFTCKELVVREGNAWLYEPNLNTLREKLHLPVGSCELAVPLKAKANWHSGNVRREAYATILHSANFYVCGAIAAAQSIRLAGSTRDLVILVDETISDYHRGGLEAAGWKIHTIKRIRNPKAEQDAYNEWNYSKFRLWQLTDYDKIIFIDADLLILRNIDFLFEMPEITAIGNNATLFNSGVMVVEPSNCTFQLLMDHINEIESYNGGDQGYLNEIFTWWHRIPKHMNFLKHYWEGDEEEKKQMKTRLFGADPPVLYVLHYLGLKPWLCFRDYDCNWNVGKLQEFASDVAHRTWWKVHDAMPDNLHKYCLLRSKQKAALEWDRREAEKANFSDGHWKIKIKDPRLETCYEEFCFWESMLWHWGETNWTDNATSSPTPPMVNTASLSSL

SEQ ID NO: 5 - DNA, 1485 bp, Arabidopsis thaliana
  CDS (1)..(1485)
atggatttacaaagaactttgatgttctcttgttgggttctgtctcttttgatcatcaaaacgacagcgtataacgagaaacagctgttccagccgcttgaaacggaaaacgcaaacgcgatgaccgcggttatggagcgaggattaaagacgcagcggcggccggagcacaagaacgcttatgcgacgatgatgtacatgggaacaccaagagactacgagttctacgttgcgacacgtgtcttgatcagatcgcttaagagtctccacgtggacgctgatatcgtcgttatagcctccctcgacgttcctatcaactggattcacgctctggaagaagaagatggagctaaagtagtgagagtagagaatcttgagaatccatacaagaaacaaaccaacttcgacaacagattcaagcttagtctaaacaagctctacgcttggtctctctctgattatgaccgtgttgtaatgcttgatgtcgacaatctctttctcaagaacaccgacgagctcttccagtgtggccaattttgtgctgtcttcatcaacccttgcatcttccacactggtctctttgtgttgcagccatcaatggaggtctttagagacatgcttcatgagcttgaagtaaagagagataaccctgatggagctgatcaaggctttcttgtcagctacttctctgatttactcaatcagcctctctttcgtcctcctcccgataaccgcaccgcgcttaagggacattttaggcttcctttgggatatcaaatggacgcatcttattactaccttaagctcagatggaacgtaccatgtggaccaaacagtgtgataacgttcccaggagcagtatggttaaagccatggtattggtggtcatggcctgttcttcctttaggcctttcatggcaccaccaacgccgctacacgattagttattcagcagagatgccttgggtcctaacccaagcagtgttctacctaggaattatactagtcacacgtctagcgcgtcccaacatgaccaagctatgttaccgacgttctgataagaatctaagcatgatccagacagctttcaagtttgttgcactcctctttatcctctcagcctacattataccattcttcatcatcccacagacgatccacccgctcattggttggtctctctacttaaccggctcctttgctctctctaccatacccatcaacgccttcttgcttcccattctccctgtcataacaccgtggcttggcattttcgggacactcctcgtgatggcttttccttcttatcctgatggcgttgttagagcattgtcggttttcgggtatgcattttgttgtgcaccgtttctatgggtctcctttgtgaagatcacatcgcatcttcagattatgattgacaaagaggttttgtttccgcggttgggtgaatccggagtcacttctggtctcagcaaattgtactga

SEQ ID NO: 6 - protein, 494 aa, Arabidopsis thaliana
MDLQRTLMFSCWVLSLLIIKTTAYNEKQLFQPLETENANAMTAVMERGLKTQRRPEHKNAYATMMYMGTPRDYEFYVATRVLIRSLKSLHVDADIVVIASLDVPINWIHALEEEDGAKVVRVENLENPYKKQTNFDNRFKLSLNKLYAWSLSDYDRVVMLDVDNLFLKNTDELFQCGQFCAVFINPCIFHTGLFVLQPSMEVFRDMLHELEVKRDNPDGADQGFLVSYFSDLLNQPLFRPPPDNRTALKGHFRLPLGYQMDASYYYLKLRWNVPCGPNSVITFPGAVWLKPWYWWSWPVLPLGLSWHHQRRYTISYSAEMPWVLTQAVFYLGIILVTRLARPNMTKLCYRRSDKNLSMIQTAFKFVALLFILSAYIIPFFIIPQTIHPLIGWSLYLTGSFALSTIPINAFLLPILPVITPWLGIFGTLLVMAFPSYPDGVVRALSVFGYAFCCAPFLWVSFVKITSHLQIMIDKEVLFPRLGESGVTSGLSKLY

SEQ ID NO: 7 - DNA, 1494 bp, Arabidopsis thaliana
  CDS (1)..(1494)
atggatttgcagagaggctttgtgttcttgtctttggttctatcttttatgataatcgaaacgacggcgtatcgagagagacagctgctgctgctgcaaccaccgcaagaaacggcgatagataccgcaaacgcggtggtgacggttcaagatcgaggtttgaagacgcggcgaccggagcataagaacgcatacgcaacgatgatgtacatggggacgccaagagactacgagttctacgttgcgacacgtgttttgatcagatcgttgagaagtcttcacgtggaagctgatctcgtcgtcatcgcttctctcgacgttcctctccgatgggttcaaaccttggaagaggaagatggagctaaagtggtgagagttgaaaatgtggataatccatacaggagacagaccaacttcaacagtagattcaagcttactctaaacaagctctacgcttgggctttgtctgattacgaccgtgtggtcatgctagatgccgataacctctttcttaagaaagccgacgagttgttccagtgtgggcgcttctgtgcggtcttcattaacccttgtatcttccacactggtctcttcgtgttgcagccatcagtggaagtgttcaaggacatgctccatgagctacaagttggaagaaagaatcctgatggagctgatcaaggttttcttgtcagttacttctctgatcttcttgaccaacctctctttagtcctccgagtaacggatctgtacttaatggtcacttgagacttcccttaggctaccaaatggacgcttcttatttctatcttaagctaagatggaacataccctgtggaccaaacagtgtgattacattcccgggagctgtttggttaaagccatggtactggtggtcatggcctgttcttccactaggtttctcatggcacgagcagcgtcgcgccactatagggtactcagccgaaatgcctttggttataatccaagcaatgttttaccttggaatcatagtggttacacgactagctcgtcctaacataaccaagctatgttatcgccgctctgaccgcaacttaacaacgatccaagctggttttaagttgatcgcgcttctctctgtagttgcagcctacatcttcccattcttcaccatccctcacactatccacccactcatcggctggtcgctctacttgatggcttcttttgctctctcttccatttcaatcaacactctcctcctcccaacgctccctgttctcactccatggctaggaattctcggcactctccttgtcatggccttcccttggtaccctgatggagtggtcagagccttgtcagttttcgcatacgcattttgttgcgcaccctttgtgtgggtttcattccgcaaaatcacatcgcacctccaggttttgattgagaaagaggtgttgttcccgcgattgggagactcaggggtcacttcaggcttcagcaaattgtattag

SEQ ID NO: 8 - protein, 497 aa, Arabidopsis thaliana
MDLQRGFVFLSLVLSFMIIETTAYRERQLLLLQPPQETAIDTANAVVTVQDRGLKTRRPEHKNAYATMMYMGTPRDYEFYVATRVLIRSLRSLHVEADLVVIASLDVPLRWVQTLEEEDGAKVVRVENVDNPYRRQTNFNSRFKLTLNKLYAWALSDYDRVVMLDADNLFLKKADELFQCGRFCAVFINPCIFHTGLFVLQPSVEVFKDMLHELQVGRKNPDGADQGFLVSYFSDLLDQPLFSPPSNGSVLNGHLRLPLGYQMDASYFYLKLRWNIPCGPNSVITFPGAVWLKPWYWWSWPVLPLGFSWHEQRRATIGYSAEMPLVIIQAMFYLGIIVVTRLARPNITKLCYRRSDRNLTTIQAGFKLIALLSVVAAYIFPFFTIPHTIHPLIGWSLYLMASFALSSISINTLLLPTLPVLTPWLGILGTLLVMAFPWYPDGVVRALSVFAYAFCCAPFVWVSFRKITSHLQVLIEKEVLFPRLGDSGVTSGFSKLY

SEQ ID NO: 9 - DNA, 1944 bp, Arabidopsis thaliana
  CDS (1)..(1944)
atgcatccagcttcatgtagtctctcactcaggaaagtcaaacttaatattcttacagtgagaatgaagctctcttctgaaagtcccatggcgccgtcaagtcagtcaagtcatcgactttacatttccagtgagaaaacaaaaacgaagagattccaaagaaacggatacactctcgatgttgaaatgtgtgtcaacttctctagtctgaaacttgttttgtttcttatgatgctggttgctatgttcacactctactgttctccaccgttgcaaattcctgaagatccatcaagttttgcaaacaaatggatactagaacctgctgtaaccacagatcctcgctatatagctacatctgagatcaactggaacagtatgtcacttgttgttgagcattacttatctggcagaagcgagtatcaaggaattggctttctaaatctcaacgataacgagattaatcgatggcaggtggtcataaaatctcactgtcagcatatagctttgcatctagaccatgctgcaagtaacataacttggaaatctttatacccggaatggattgacgaggaagaaaaattcaaagtccccacttgtccttctcttccttggattcaagttcctgacaagtctcgaatcgatcttatcattgccaagctcccatgtaacaagtcaggaaaatggtcaagagatgtggctagattgcacttacaacttgcagcagctcgagtggcggcatcttctgaagggcttcatgatgttcatgtgattttggtatcagattgctttccaataccgaatctttttacgggtcaagaacttgttgcccgtcaaggaaacatatggctgtataagcctaaacttcaccagttaagacaaaagttacaacttcctgttggttcctgtgaactttctgttcctcttcaagctaaagataatttctactcggcaaatgccaagaaagaagcgtacgcgacgatcttgcactcagatgatgcttttgtctgtggagccattgcagtagcacagagcattcgaatgtcaggctctactcgcaatttggtaatactagtcgatgattcgatcagtgaataccatagaagtggcttggaatcagctggatggaagattcacacatttcaaagaatcagaaacccgaaagctgaagcaaatgcatataaccaatggaactacagcaaattccgtctttgggaattgacagaatacaacaagatcatcttcattgatgcagacatgcttatcctcagaaacatggatttcctcttcgagtaccccgaaatctccacaactggaaacgacggtacgctcttcaactccggtctaatggtgattgaaccatcaaattcaacattccagttactaatggatcacatcaacgatatcaattcctacaatggaggagaccaaggttaccttaacgagatattcacatggtggcatcggattccaaaacacatgaatttcttgaagcatttctgggaaggagacacacctaagcacaggaaatctaagacgagactatttggagctgatcctccgatactctacgttcttcattacctaggttacaacaaaccatgggtatgcttcagagactacgattgcaattggaatgtcgttggataccatcaattcgcgagcgatgaagcacacaaaacttggtggagagtgcacgacgcgatgcctaagaaattgcagaggttttgtctactgagttcgaaacaaaaggcgcaacttgagtgggatcggagacaagctgagaaagcgaattacagagacggacattggaggattaagatcaaagataagagacttacgacttgttttgaagatttctgtttctgggagagtatgctttggcattggggtgagactcagaccaactccaccgtcgctgctgattcttcctccaccgcgtaa

SEQ ID NO: 10 - protein, 647 aa, Arabidopsis thaliana
MHPASCSLSLRKVKLNILTVRMKLSSESPMAPSSQSSHRLYISSEKTKTKRFQRNGYTLDVEMCVNFSSLKLVLFLMMLVAMFTLYCSPPLQIPEDPSSFANKWILEPAVTTDPRYIATSEINWNSMSLVVEHYLSGRSEYQGIGFLNLNDNEINRWQVVIKSHCQHIALHLDHAASNITWKSLYPEWIDEEEKFKVPTCPSLPWIQVPDKSRIDLIIAKLPCNKSGKWSRDVARLHLQLAAARVAASSEGLHDVHVILVSDCFPIPNLFTGQELVARQGNIWLYKPKLHQLRQKLQLPVGSCELSVPLQAKDNFYSANAKKEAYATILHSDDAFVCGAIAVAQSIRMSGSTRNLVILVDDSISEYHRSGLESAGWKIHTFQRIRNPKAEANAYNQWNYSKFRLWELTEYNKIIFIDADMLILRNMDFLFEYPEISTTGNDGTLFNSGLMVIEPSNSTFQLLMDHINDINSYNGGDQGYLNEIFTWWHRIPKHMNFLKHFWEGDTPKHRKSKTRLFGADPPILYVLHYLGYNKPWVCFRDYDCNWNVVGYHQFASDEAHKTWWRVHDAMPKKLQRFCLLSSKQKAQLEWDRRQAEKANYRDGHWRIKIKDKRLTTCFEDFCFWESMLWHWGETQTNSTVAADSSSTA

SEQ ID NO: 11 - DNA, 1857 bp, Arabidopsis thaliana
  CDS (1)..(1857)
atgataccttcctcaagtcccatggagtcaagacatcgactctcgttctcaaaagagaagacaagtaggaggagatttcaaagaattgagaagggtgtcaagttcaacactctgaaacttgtgttgatttgtataatgcttggagctttgttcacgatctaccgttttcgttatccaccgctacaaattcctgaaattccaactagttttggtcttactactgatcctcgctatgtagctacagctgagatcaactggaaccatatgtcaaatcttgttgagaagcacgtatttggtagaagcgagtatcaaggaattggtcttataaatcttaacgataacgagattgatcgattcaaggaggtaacgaaatctgactgtgatcatgtagctttgcatctagattatgctgcaaagaacataacatgggaatctttatacccggaatggattgatgaagttgaagaattcgaagtccctacttgtccttctctgcctttgattcaaattcctggcaagcctcggattgatcttgtaattgccaagcttccgtgtgataaatcaggaaaatggtctagagatgtggctcgcttgcatttacaacttgcagcagctcgagtggcggcttcttctaaaggacttcataatgttcatgtgattttggtatctgattgctttccaataccgaatctttttacgggtcaagaacttgttgcccgtcaaggaaacatatggctgtataagcctaatcttcaccagctaagacaaaagttacagcttcctgttggttcctgtgaactttctgttcctcttcaagctaaagataatttctactccgcaggtgcaaagaaagaagcttacgcgactatcttgcattctgcccaattttatgtctgtggagccattgcagctgcacagagcattcgaatgtcaggctctactcgtgatctggtcatacttgttgatgaaacgataagcgaataccataaaagtggcttggtagctgctggatggaagattcaaatgtttcaaagaatcaggaacccgaatgctgtaccaaatgcctacaacgaatggaactacagcaagtttcgtctttggcaactgactgaatacagtaagatcatcttcatcgatgcagacatgcttatcctgagaaacattgatttcctcttcgagttccctgagatatcagcaactggaaacaatgctacgctcttcaactctggtctaatggtggttgagccatctaattcaacattccagttactaatggataacattaatgaagttgtgtcttacaacggaggagaccaaggttaccttaacgagatattcacatggtggcatcggattccaaaacacatgaatttcttgaagcatttctgggaaggagacgaacctgagattaaaaaaatgaagacgagtctatttggagctgatcctccgatcctatacgttcttcattacctaggttataacaaaccctggttatgcttcagagactatgactgcaattggaatgtcgatattttccaggaatttgctagtgacgaggctcataaaacctggtggagagtgcacgacgcaatgcctgaaaacttgcataagttctgtctactaagatcgaaacagaaggcgcaacttgaatgggataggagacaagcagagaaagggaactacaaagatggacattggaagataaagatcaaagacaagagacttaagacttgtttcgaagatttctgcttttgggagagtatgctttggcattggggtgagacgaactctaccaacaattcttccaccaccaccacttcatcaccgccgcataaaaccgctctcccttccctgtga

SEQ ID NO: 12 - protein, 618 aa, Arabidopsis thaliana
MIPSSSPMESRHRLSFSKEKTSRRRFQRIEKGVKFNTLKLVLICIMLGALFTIYRFRYPPLQIPEIPTSFGLTTDPRYVATAEINWNHMSNLVEKHVFGRSEYQGIGLINLNDNEIDRFKEVTKSDCDHVALHLDYAAKNITWESLYPEWIDEVEEFEVPTCPSLPLIQIPGKPRIDLVIAKLPCDKSGKWSRDVARLHLQLAAARVAASSKGLHNVHVILVSDCFPIPNLFTGQELVARQGNIWLYKPNLHQLRQKLQLPVGSCELSVPLQAKDNFYSAGAKKEAYATILHSAQFYVCGAIAAAQSIRMSGSTRDLVILVDETISEYHKSGLVAAGWKIQMFQRIRNPNAVPNAYNEWNYSKFRLWQLTEYSKIIFIDADMLILRNIDFLFEFPEISATGNNATLFNSGLMVVEPSNSTFQLLMDNINEVVSYNGGDQGYLNEIFTWWHRIPKHMNFLKHFWEGDEPEIKKMKTSLFGADPPILYVLHYLGYNKPWLCFRDYDCNWNVDIFQEFASDEAHKTWWRVHDAMPENLHKFCLLRSKQKAQLEWDRRQAEKGNYKDGHWKIKIKDKRLKTCFEDFCFWESMLWHWGETNSTNNSSTTTTSSPPHKTALPSL

SEQ ID NO: 13 - DNA, 2130 bp, Arabidopsis thaliana
  CDS (1)..(1980)
atggcaaactctcccgctgctcctgcacccaccaccacaaccggtggtgactcccggcgacgcctctccgcgtccatagaagcaatatgcaagaggagattccggagaaatagcaaaggaggtggcagatcggatatggtgaaaccgtttaatatcataaatttttcgacacaagacaaaaacagtagttgttgttgtttcaccaagtttcagatcgtgaagcttctcttgtttatccttctctctgccactctcttcaccattatctattctcctgaagcttatcatcattctctttcccactcatcttctcgatggatatggagaagacaagatccacgttacttctcggatctggatataaactgggacgatgtgactaaaacccttgagaacatcgaagaaggccgtacgatcggtgtcttgaattttgattcgaacgagatccaacgatggagagaagtatccaagagcaaggacaatggggatgaagaaaaagttgttgtattgaatctagattacgcagacaagaatgtgacttgggacgcactatatccagagtggatcgatgaggagcaagaaacagaggtccctgtttgtcctaatatcccgaacattaaggtacctacaagaagactcgatctgatcgtcgtgaaacttccttgtcggaaagaagggaattggtcgagagacgtcgggagattgcatctacagctagcggctgcaactgtggcggcttcggccaaagggtttttcaggggacatgtgttttttgtatctagatgctttccgattccgaatcttttccggtgtaaagatcttgtgtctcggagaggcgatgtttggttgtacaaacctaatcttgataccttgagagacaagcttcagctgcctgtagggtcttgtgagctatctcttcctcttggcatccaagataggccaagcttaggaaaccctaaaagagaagcttacgcaacaattcttcactcagctcacgtttacgtctgcggtgcaatcgccgcggctcagagcataagacagtctggttcgacgagagaccttgttatccttgttgatgacaacatcagcggttaccaccggagtggactagaagccgcgggttggcaaatccggacgatacagaggattcgaaaccctaaggcagagaaagatgcttacaacgaatggaactacagcaagttccggctatggcagctgactgattacgacaaaatcattttcatcgacgcggatctcttaatcttgagaaacatcgatttcttgttctcgatgcctgagatctcagctacaggaaacaatggaactctgtttaattcaggagttatggtgatcgagccttgcaactgtacgtttcagcttctgatggaacatataaacgagattgagtcttataacggtggagatcaaggttacttaaacgaggtattcacatggtggcaccggattccaaaacatatgaatttcttgaagcatttttggattggcgatgaagatgacgcgaaacgcaagaaaacagagctttttggagcagagcctcctgttctttatgttcttcattaccttgggatgaagccgtggttatgttaccgtgactacgactgtaacttcaactccgacatattcgttgagtttgctaccgatatcgctcatcgaaaatggtggatggtccacgacgccatgccacaggaacttcaccaattctgttacttgcgatccaagcaaaaggcacagctggaatatgatcgccggcaagcagaggccgcaaattatgccgacggtcattggaaaataagagtaaaggacccgagattcaaaatttgcatcgacaaattatgtaattggaaaagtatgctgcggcattggggcgaatcaaattggactgactacgagtcttttgttcccaccccaccagccattaccgtagaccggagatcatcacttcccggccataacttgtgacgcaataattatacatacttattaatggatttcatgagttttttggtttgaattgttgctgcgagattaggtgaatatcagttgtgtaactatatctttttcctatagtttgttcaaattgaataaaacatttttttgcagtttaaccac

SEQ ID NO: 14 - protein, 659 aa, Arabidopsis thaliana
MANSPAAPAPTTTTGGDSRRRLSASIEAICKRRFRRNSKGGGRSDMVKPFNIINFSTQDKNSSCCCFTKFQIVKLLLFILLSATLFTIIYSPEAYHHSLSHSSSRWIWRRQDPRYFSDLDINWDDVTKTLENIEEGRTIGVLNFDSNEIQRWREVSKSKDNGDEEKVVVLNLDYADKNVTWDALYPEWIDEEQETEVPVCPNIPNIKVPTRRLDLIVVKLPCRKEGNWSRDVGRLHLQLAAATVAASAKGFFRGHVFFVSRCFPIPNLFRCKDLVSRRGDVWLYKPNLDTLRDKLQLPVGSCELSLPLGIQDRPSLGNPKREAYATILHSAHVYVCGAIAAAQSIRQSGSTRDLVILVDDNISGYHRSGLEAAGWQIRTIQRIRNPKAEKDAYNEWNYSKFRLWQLTDYDKIIFIDADLLILRNIDFLFSMPEISATGNNGTLFNSGVMVIEPCNCTFQLLMEHINEIESYNGGDQGYLNEVFTWWHRIPKHMNFLKHFWIGDEDDAKRKKTELFGAEPPVLYVLHYLGMKPWLCYRDYDCNFNSDIFVEFATDIAHRKWWMVHDAMPQELHQFCYLRSKQKAQLEYDRRQAEAANYADGHWKIRVKDPRFKICIDKLCNWKSMLRHWGESNWTDYESFVPTPPAITVDRRSSLPGHNL

SEQ ID NO: 15 - DNA, 15294 bp, Artificial Sequence
  misc_feature (1)..(15294): Description of Artificial Sequence vector
ggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccaagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgatctgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtaccatcatgttacaaacttttttgctgtgagcagtagatatggaaacccggaggacctaaagtatctgatagataaagcacatagcttgggtttacaggttctggtggatgtagttcacagtcatgcaagcaataatgccactgatggcctcaatggctttgatattggccaaggttctcaagaatcctactttcatgctggagagcaagggtaccataagttgtgggatagcaggctgttcaactatgccaattgggaggttcttcgtttccttctttccaacttgaggtggtggctagaagagtataactttgacggatttcgatttgatggaataacttctatgctgtatgttcatcatggaatcaatatgggatttacaggaaactataatgagtatttcagcgaggctacagatgttgatgctgtggtctatttaatgttggccaataatctgattcacaagattttcccagacgcaactgttattgccgaagatgtttctggtatgccgggccttggccggcctgtttctgagggaggaattggttttgattaccgcctggcaatggcaatcccagataagtggatagattatttaaagaataaaaatgatgaagattggtccatgaaggaagtaacatcgagtttgacaaataggagatatacagagaagtgtatagcatatgcggagagccatgatcagtctattgtcggtgacaagaccattgcatttctcctaatggacaaagagatgtattctggcatgtcttgcttgacagatgcttctcctgttattgatcgaggaattgcgcttcacaagatgatccattttttcacaatggccttgggaggagaggggtacctcaatttcatgggtaacgagtttggccatcctgagtggattgacttccctagagagggcaataattggtgttatgacaaatgtagacgccagtggaaccttgcggatagcgaacacttgagatacaagtttatgaatgcatttgatagagctatgaattcgctcgatgaaaagttctcattcctcgcatcaggaaaacagatagtaagcagcatggatgatgagaagaaggttgttgtgtttgaacgtggtgacctggtatttgtattcaacttccacccaaataacacatacgaagggtataaagttggatgtgacttgccagggaagtacagagttgcactggacagtgatgcttgggaatttggtggccatggaagagctggtcatgatgttgaccatttcacatcaccagaaggaatacctggagttccagaaacaaatttcaatggtcgtccaaattccttcaaagtgctgtctcctgcgcgaacatgtgtggcttattacagagttgacgaacgcatgtcagaaactgaagtttaccagacagacatttctagtgagctactaccaacagccaatatcgaggagagtgacgagaaacttaaagattcgttatctacaaatatcagtaacgttgacgaactcatgtcagaaactgaagtttaccagacagacatttctagtgagctactaccaacagccagtatcgaggagagtgacgagaaacttaaagattcattatctacaaatatcagtacgtggttatcattggatgtgggattcccgcctctttaattatggaaactgggaggtacttaggtatcttctctcaaatgcgagatggtggttggatgagttcaaatttgatggatttagattcgatggtgtgacatcaataatgtatactcaccacggattatcggtgggattcactgggaactacaaggaatactttggactcgcaactgatgtggatgctgttgtgtatctgatgctggtcaacgatcttattcatgggcttttccagatgcaattaccattggtgaagatgttagcggaatgccgacattttgtattcccgttcaagatgggggtgttggctttgactatcggctgcatatggcaattgctgataaatggattgagttgctcaagaaacgggatgaggattggagagtgggtgatattgttcatacactgacaaatagaagatggtcggaaaagtgtgtttcatacgctgaaagtcatgatcaagctctagtcggtgataaaactatagcattctggctgatggacaaggatatgtatgattttatggctctggatagaccatcaacatcattaatagatcgtgggatagcattgcacaagatgattaggcttgtaactatgggattaggaggagaagggtacctaaatttcatgggaaatgaattcggccaccctgagtggattgatttccctagggctgaacaacacctctctgatggctcagtaattcccggaaaccaattcagttatgataaatgcagacggagatttgacctgggagatgcagaatatttaagataccgtgggttgcaagaatttgaccgggctatgcagtatcttgaagataaatatgagtttatgacttcagaacaccagttcatatcacgaaaggatgaaggagataggatgattgtatttgaaaaaggaaacctagtttttgtctttaattttcactggacaaaaagctattcagactatcgcataggctgcctgaagcctggaaaatacaaggttgccttggactcagatgatccactttttggtggcttcgggagaattgatcataatgccgaatatttcacctttgaaggatggtatgatgatcgtcctcgttcaattatggtgtatgcacctagtagaacagcagtggtctatgcactagtagacaaagaagaagaagaagaagaagaagtagcagtagtagaagaagtagtagtagaagaagaatgaacgaacttgtgatcgcgttgaaagatttgaaggctacatagctctagagtcgacctgcatgaaatcagaaataattggaggagatgagtaaaagttaccacttgttgagctgtgtgagtgagtgagtgagaatgaggaggtgcctgccttatttgtagcaggtttcagtgacacgtgtcaagagaatagcgggtggctatcccttagcagaaggcaactgtggacactgtattatagggaaatgctcatcgacagtattatgggccctctctttgttgattcacggctggacttcaacttgggccttgcaatgggcccgtccggttctgtctcctagtatctaaaaaactaaaccaactccctcctaccgctaccacttgacattcctatgtctcgtgttaattaaattattattatagtaattaaaaataatatctaggtactggtactggtccctccctccactagaatattagttacttcccccttagctttgtattccaaattactgtaaatatattttctaattttttacgacaaacaagatctaattatgaatgcacaattctaaaggttgaatacattactttacttggtttagcctatattaagttgcattttagtattaagattgagatgcatggttctattacaaaattgatacactgctaaaggaaggatggttaaaaacaacattcaatgtttgttacatttcttcctattgtattttttttttaacgagcttcccgtatacatcataacatgtctccgttccacttggcaggaaaaaaaaatacccaaacaggaagatactgtcaagtatatccatagatgaggacttaatggataggcttttcgaggattcataaatcataatatctggcggaggagtcaattaaatacttgtggtttgtatcctgattactccgtcaacagccaaatagaaaagtttgaaaagagagaaaggatttggtacaagatactgttgcatttgttaagtaatgaacaaaacggagtaacataattttctatctcgttaaagcttcacgctgccgcaagcactcagggcgcaagggctgctaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctcgactctagtcgagggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctagcttcgacggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcg

SEQ ID NO: 16 - DNA, 2019 bp, Arabidopsis thaliana
  CDS (1)..(2010)
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatattctcgtcgaggctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatattcctaggattattgaggaggctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaactctagcgatgaattgggtaggtttgttgagcttacgggaatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaaacttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaaacttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagttttataatgaatgtgcaagagctagccactattcgtgtagagaatcttccagtgaaggtacttttattaaacaaccagcatcttggcatggttatgcaatgggaagatcggttctacaaagctaaccgagcacacacatttctcggagatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagctc

SEQ ID NO: 17 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPNGGTFNDVITEGDGRIKY

SEQ ID NO: 18 - DNA, 259 bp, Artificial Sequence
  promoter (1)..(259): Description of Artificial Sequencebe2promoter fragment
gatctctaaataattcgaaatatctttgttattatttttttctattcaaattgcaattagacataagtcattttaactgaagctgcattgatgaaaaattatactatgtctttatgtatatatattaatgttttaaattcctttatagtgataaagatggttcgaaacatgctacaaattattatacgaagttactttttttaatctactttaacaattttctaatttcactattgaacatagataccagcccgggccg

SEQ ID NO: 19 - DNA, 400 bp, Artificial Sequence
  misc_feature (1)..(400): Description of Artificial Sequence RNAi420be2be1
gaattgttgttctcatggacatcgttcacagccatgcatcaaataatactttagatggactgaacatgtttgacggcaccgatagttgttactttcactctggagctcgtggttatcattggatgtgggattcccgcctctttaactatggaaactgggaggtacttaggtatcttctctcaaatgcgagatggtggttgccatttcacatcaccagaaggaatacctggagttccagaaacaaatttcaatggtcgtccaaattccttcaaagtgctgtctcctgcgcgaacatgtgtggcttattacagagttgacgaacgcatgtcagaaactgaagtttaccagacagacatttctagtgagctactaccaacagccaatatcgaggagagtgacg

SEQ ID NO: 20 - DNA, 1105 bp, Artificial Sequence
  misc_feature (1)..(1105): Description of Artificial SequenceRNAifragment
gaattgttgttctcatggacatcgttcacagccatgcatcaaataatactttagatggactgaacatgtttgacggcaccgatagttgttactttcactctggagctcgtggttatcattggatgtgggattcccgcctctttaactatggaaactgggaggtacttaggtatcttctctcaaatgcgagatggtggttgccatttcacatcaccagaaggaatacctggagttccagaaacaaatttcaatggtcgtccaaattccttcaaagtgctgtctcctgcgcgaacatgtgtggcttattacagagttgacgaacgcatgtcagaaactgaagtttaccagacagacatttctagtgagctactaccaacagccaatatcgaggagagtgacgatcaagctgatctctaaataattcgaaatatctttgttattatttttttctattcaaattgcaattagacataagtcattttaactgaagttgcattgatgaaaaattatactatgttttatgtatatatattaatttttaaattcctttatagtgataaagatagttcgaaacatgctataaattattatacgaatttacgttactttttttaatctactttaacaattttctaatttcactattgaacatagataccagcccgggccgtcgacctcgaattcgcccttggagagtgacgttcgcgtcactctcctcgatattggctgttggtagtagctcactagaaatgtctgtctggtaaacttcagtttctgacatgcgttcgtcaactctgtaataagccacacatgttcgcgcaggagacagcactttgaaggaatttggacgaccattgaaatttgtttctggaactccaggtattccttctggtgatgtgaaatggcaaccaccatctcgcatttgagagaagatacctaagtacctcccagtttccatagttaaagaggcgggaatcccacatccaatgataaccacgagctccagagtgaaagtaacaactatcggtgccgtcaaacatgttcagtccatctaaagtattatttgatgcatggctgtgaacgatgtccatgagaacaacaattc

SEQ ID NO: 21 - DNA, 180 bp, Artificial Sequence
  misc_feature (1)..(180): Description of Artificial SequenceSBE RNAi 1
actagtggtacttaggtatcttctctcaaatgcgagatggtggttgagtgagctactaccaacagccaatatcgaggagagtgacgttcgcgtcactctcctcgatattggctgttggtagtagctcactcaaccaccatctcgcatttgagagaagatacctaagtaccttttggtacc

SEQ ID NO: 22 - DNA, 420 bp, Artificial Sequence
  misc_feature (1)..(420): Description of Artificial SequenceSBE RNAi 2
actagttggagctcgtggttatcattggatgtgggattcccgcctctttaactatggaaactgggaggtacttaggtatcttctctcaaatgcgagatggtggttggcttattacagagttgacgaacgcatgtcagaaactgaagtttaccagacagacatttctagtgagctactaccaacagccaatatcgaggagagtgacgttcgcgtcactctcctcgatattggctgttggtagtagctcactagaaatgtctgtctggtaaacttcagtttctgacatgcgttcgtcaactctgtaataagccaaccaccatctcgcatttgagagaagatacctaagtacctcccagtttccatagttaaagaggcgggaatcccacatccaatgataaccacgagctccattttggtacc

SEQ ID NO: 23 - DNA, 37 bp, Artificial Sequence
  misc_feature (1)..(37): Description of Artificial Sequencespacer
atcaagcttatcgataccgtcgacctcgaagcttgat

SEQ ID NO: 24 - DNA, 837 bp, Artificial Sequence
  misc_feature (1)..(837): Description of Artificial Sequencefragment of be2 and be1 in pHAS3 for RNAi
gaattgttgttctcatggacatcgttcacagccatgcatcaaataatactttagatggactgaacatgtttgacggcaccgatagttgttactttcactctggagctcgtggttatcattggatgtgggattcccgcctctttaactatggaaactgggaggtacttaggtatcttctctcaaatgcgagatggtggttgccatttcacatcaccagaaggaatacctggagttccagaaacaaatttcaatggtcgtccaaattccttcaaagtgctgtctcctgcgcgaacatgtgtggcttattacagagttgacgaacgcatgtcagaaactgaagtttaccagacagacatttctagtgagctactaccaacagccaatatcgaggagagtgacgatcaagcttatcgataccgtcgacctcgaagcttgatcgtcactctcctcgatattggctgttggtagtagctcactagaaatgtctgtctggtaaacttcagtttctgacatgcgttcgtcaactctgtaataagccacacatgttcgcgcaggagacagcactttgaaggaatttggacgaccattgaaatttgtttctggaactccaggtattccttctggtgatgtgaaatggcaaccaccatctcgcatttgagagaagatacctaagtacctcccagtttccatagttaaagaggcgggaatcccacatccaatgataaccacgagctccagagtgaaagtaacaactatcggtgccgtcaaacatgttcagtccatctaaagtattatttgatgcatggctgtgaacgatgtccatgagaacaacaattc

SEQ ID NO: 25 - DNA, 24 bp, Artificial sequence
  misc_feature (1)..(24): Npt2_for primer
agcaaggtgagatgacaggagatc

SEQ ID NO: 26 - DNA, 22 bp, Artificial sequence
  misc_feature (1)..(22): Npt2_rev primer
cagacaatcggctgctctgatg

SEQ ID NO: 27 - DNA, 22 bp, Artificial sequence
  misc_feature (1)..(22): AHAS1_frw primer
aacaacaacatcttcttcgatc

SEQ ID NO: 28 - DNA, 20 bp, Artificial sequence
  misc_feature (1)..(20): AHAS1_rev primer
taacgagatttgtagctccg

SEQ ID NO: 29 - DNA, 20 bp, Artificial sequence
  misc_feature (1)..(20): StGH1 sense RNA primer
tgaagacagcacaaaactgg

SEQ ID NO: 30 - DNA, 20 bp, Artificial sequence
  misc_feature (1)..(20): StGH1 antisense RNA primer
gtgaaagtttgaacgcacac

SEQ ID NO: 31 - DNA, 20 bp, Artificial sequence
  misc_feature (1)..(20): StGH2 sense RNA primer
agtgccataacatgctttcc

SEQ ID NO: 32 - DNA, 21 bp, Artificial sequence
  misc_feature (1)..(21): StGH2 antisense RNA primer
cacatttcagctgttgatgga

SEQ ID NO: 33 - DNA, 21 bp, Artificial sequence
  misc_feature (1)..(21): StGH1 forward primer
tcgagtcgccacgtagaactc

SEQ ID NO: 34 - DNA, 23 bp, Artificial sequence
  misc_feature (1)..(23): StGH1 reverse primer
gaaatgcgtatgcgactatgatg

SEQ ID NO: 35 - DNA, 16 bp, Artificial sequence
  misc_feature (1)..(16): StGH1 TaqMan probe
agtctctcggagttcc

SEQ ID NO: 36 - DNA, 22 bp, Artificial sequence
  misc_feature (1)..(22): StGH2 forward primer
ggtgctgatcctccagttctct

SEQ ID NO: 37 - DNA, 22 bp, Artificial sequence
  misc_feature (1)..(22): StGH2 reverse primer
gtccctgaagcataaccaaggt

SEQ ID NO: 38 - DNA, 19 bp, Artificial sequence
  misc_feature (1)..(19): StGH2 TaqMan probe
ttctgcactacttaggcct

(19) United States
(12) Patent Application Publication
(10) Pub. No.: US 2006/0162021 A1
(43) Pub. Date: Jul. 20, 2006

(54) Enhanced amylose production in plants

(75) Inventors: Per Hofvander, FALSTERBO (SE); Mariette Andersson, Lund (SE)

Correspondence Address: CONNOLLY BOVE LODGE & HUTZ, LLP, P O BOX 2207, WILMINGTON, DE, 19899, US

(73) Assignee: BASF PLANT SCIENCE GMBH, LUDWIGSHAFEN (DE)

(21) Appl. No.: 10/547,660

(22) PCT Filed: Mar. 3, 2004
(86) PCT No.: PCT/EP04/02096

§ 371 Date: Sep. 1, 2005

(30) Foreign Application Priority Data
Mar. 7, 2003 (EP) 03005181.7

(51) Int. Cl. A01H 1/00; C12N 15/82; C12N 5/04

(52) U.S. Cl. 800/284; 435/468; 435/419

(57) ABSTRACT

The invention relates to methods for increasing the amylose content in plants, preferably in potato plants, by expressing a starch biosynthesis enhancing protein. The invention furthermore relates to an expression cassette expressing the polypeptide in potato plants, preferably in the tubers, the transgenic plants expressing the polypeptide and to the use of said transgenic plants for the production of fine chemicals especially other than native starches.

The present invention relates to new starch biosynthesis enhancing proteins, nucleic acids encoding a starch biosynthesis enhancing protein, a method for producing amylose with high efficiency by culturing genetically modified plants with an increased amylose biosynthesis compared to the wild type or to the genetically modified plants themselves as well as the use of these transgenic plants over-expressing at least one of the starch biosynthesis enhancing proteins for the production of amylose.

Starch is the major storage carbohydrate of plants and is mainly accumulated in seeds and tubers, which are then the reproductive tissues of plants that form those types of organs. Starch is also accumulated on a diurnal basis where starch is built up in green tissue from photosynthetic products and then metabolised for energy during the dark period. The storage starch is assembled into semi crystalline granules. Amylopectin and amylose are the two constituent molecules of starch. Amylopectin is a branched molecule consisting of linear α-1,4 glucan chains linked by α-1,6 bonds. Amylose consists essentially of the linear α-1,4 glucan chains.

Starch is utilised for many applications within the technical industry as well as the food industry. Main crops used by starch processors are maize and potato. For potato specific varieties are utilised for starch production that have been bred for high starch contents. This means that the starch content and yield is an important economic driver for the starch processing industry. A greater part of produced dry starch is used for paper production. The specifications and requirements for the starch component varies from application to application and starch is many times chemically modified in order to provide desired properties to an application. Another way to achieve starch of different qualities is to take advantage of mutations in the starch biosynthesis and more recently by genetic modification of pathways leading to starch. The first main modifications have been to separate the production of the two starch components amylopectin and amylose into different varieties. Waxy or “amylose free” varieties contain solely amylopectin type starch while there are also high amylose genotypes such as “amylose extender” in maize.

Amylose starch has several potential industrial uses as a film former or for expanded products. High amylose starch can be achieved in potatoes and other starch containing plants by inhibition of starch branching enzymes. This leads then to the concomitant reduction or elimination of amylopectin branching and thereby an increased amylose fraction.

U.S. Pat. No. 5,856,467 describes the genetically engineered modification of potato for suppressing formation of amylopectin-type starch. The document describes an antisense construct for inhibiting, to a varying extent, the expression of the gene coding for formation of starch branching enzyme (SBE gene) in potato, said antisense construct comprising a tuber specific promoter, transcription start and the first exon of the SBE gene, inserted in the antisense direction.

U.S. Pat. No. 6,169,226 relates to an amino acid sequence of a second starch branching enzyme (SBE II) of potato and a fragment thereof as well as to the corresponding isolated DNA sequences. It describes the production of transgenic potatoes and the use of these transgenic potatoes for the production of amylose-type starch.

WO 97/20040 and WO 98/20145 describe methods of altering the amylopectine/amylose starch content of plant cells by introducing into the plant cells nucleic acid sequences operably linked in sense or antisense orientation to a suitable promoter which homologous genes encodes polypeptides having SBE I or SBE II activity.

A side effect of the amylose overproduction is a decreased total starch content in the potatoes. This decrease becomes more pronounced as the amylose fraction is increased.

Basic enzymes for the production of amylopectin and amylose are starch synthases that build the linear α-1,4 glucan chains and branching enzymes breaking the α-1,4 glucan chain and reattaching them by α-1,6 bonds. Several other enzymes are likely to affect starch structure and composition, such as debranching enzymes, but initially most focus has been towards affecting the expression of starch synthases and starch branching enzymes. This has led to an extensive dissection of what enzymes are important for what features of starch synthesis. However it has never been convincingly shown how the synthesis of starch in plants whether amylose or amylopectin is initiated.

Suggestions on the initiation of starch biosynthesis have been the subject of several scientific papers since it has been difficult to attribute a primer independent function to starch synthases under other than artificial in vitro conditions. By primer independent function implies the formation of new α-1,4 glucan chains with ADP-glucose as the sole starting point and building block. One proposed pathway has been that the presence of maltooligosaccharides act as primers for the addition of further glucose units by starch synthases although it has been debated on whether concentrations are sufficient to provide the basis for starch synthesis and also how these maltooligosaccharides would be formed in the plastids.

Starch is in plants synthesised as an energy storage molecule. Much is known about the enzymes participating in the starch biosynthesis although, the initiation of the starch molecule has remained unsolved. In mammalians and yeast an energy storage molecule very similar to starch is synthesised, glycogen. The enzymatic steps for synthesis of the respective molecules are analogous. In glycogen biosynthesis the initiation of the molecule is known and synthesised by the enzyme glycogenin. Glycogenin is a self-glucosylating enzyme polymerising a linear chain of approximately 8 glucose molecules on itself. The primer of about 8 glucose residues is necessary for the enzymes catalysing the continuation of glucose incorporation to the glycogen molecule to function.

Cheng et al., 1995, Mol. and Cell. Biol. 6632-6640 compare the two yeast proteins with rabbit muscle glycogenin.

Roach et al., 1997, Progress in Nucleic Acid Research and Molecular Biology Vol 57, describe self glycosylating initiator proteins and their roll in glycogen biosynthesis.

Mu et al., 1997, Journal of Biological Chemistry 272 (44), 27589-27597 compare mammalian with yeast and C. elegans glycogenins.

Factors important for starch quantity have been investigated and many initiatives have been taken, especially in potato, to increase starch formation and content by over-expression or inhibition of various enzyme activities in areas of increased substrate supply, increased biosynthesis activity or shutting down substrate diverting pathways but so far this has led only to limited success with no commercial applications and only some scientific publications.

Regierer, B. et al., Starch content and yield increase as a result of altering adenylate pools in transgenic plants. Nat Biotechnol. 20(12):1256-60, (2002).

Sweetlove, L J et al., Starch synthesis in transgenic potato tubers with increased 3-phosphoglyceric acid content as a consequence of increased 6-phosphofructokinase activity. Planta 213(3):478-82 (2001).

Veramendi, J et al., Antisense repression of hexokinase 1 leads to an overaccumulation of starch in leaves of transgenic potato plants but not to significant changes in tuber carbohydrate metabolism. Plant Physiol. 121(1):123-34 (1999).

Geigenberger, P et al., Overexpression of pyrophosphatase leads to increased sucrose degradation and starch synthesis, increased activities of enzymes for sucrose-starch interconversions, and increased levels of nucleotides in growing potato tubers. Planta. 205(3):428-37(1998).

Sweetlove, L J et al., Starch metabolism in tubers of transgenic potato (Solanum tuberosum) with increased ADPglucose pyrophosphorylase. Biochem J. 320 (2):493-8 (1996).

In other research a biochemical function superficially similar to the one initiating glycogen production in animals was investigated. A class of genes have then been isolated from several plants and was given the name amylogenin (WO94/04693; Sing, D. et al, β-Glucosylarginin: a new glucose-protein bond in a self-glucosylating protein from sweet corn, FEBS Letters 376:61-64, (1995) in the belief that it was the plant equivalent of glycogenin which acts as a self-glycosylating enzyme and provide primers for starch biosynthesis in plants. These genes have no resemblance from a structural point of view to the genes coding for glycogenin and have later been determined not to have a function in starch biosynthesis but rather might be of importance for cell wall formation, see Bocca, S. N et al., Molecular cloning and characterization of the enzyme UDP-glucose: protein transglucosylase from potato. Plant Physiology and Biochemistry 37(11):809-819(1999).

WO 98/50553 describes nucleic acid fragments encoding a plant glycogenin or a water stress protein. WO 98/50553 also relates to the construction of chimeric genes encoding all or a portion of a plant glycogenin in sense or antisense orientation, wherein expression of the chimeric gene results in production of altered levels of a plant glycogenin in a transformed host cell.

Thus although many enzymes and pathways have been investigated in plants, the question on how starch formation is initiated and what determines the starch content is still unresolved.

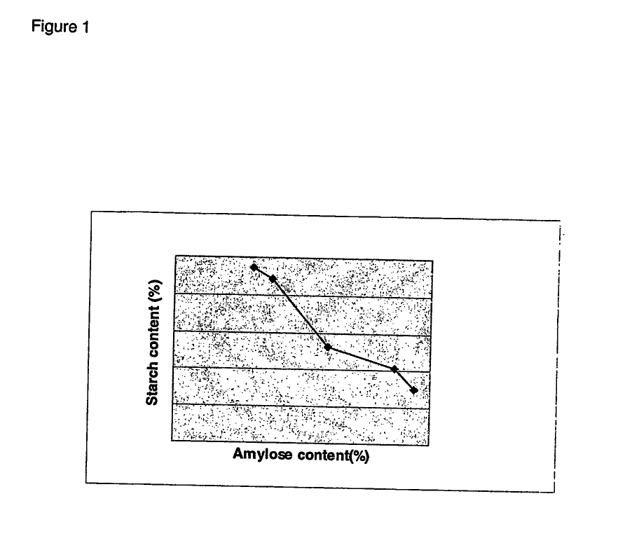

Amylose is a commercially important starch product with many uses but unfortunately an increase in amylose content in transgenic potato plants is associated with a significant decrease in starch content, see FIG. 1.

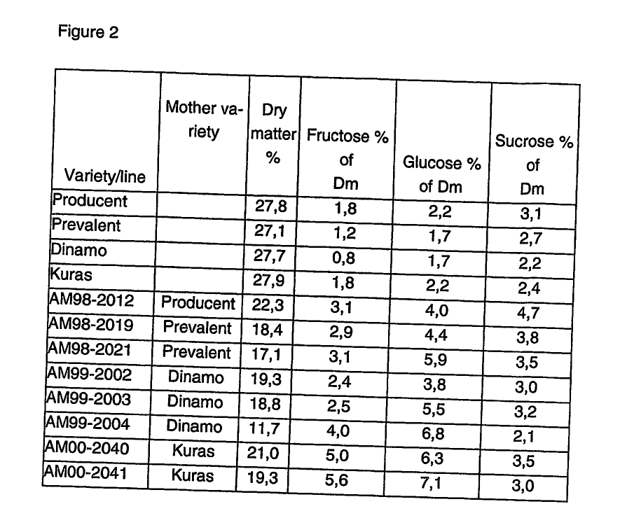

Analyses of transgenic high amylose potato lines show that there is an excess of soluble sugars in these lines, see FIG. 2. This indicates that the starch biosynthesis in these transgenic lines is not efficient enough for incorporation of available sugars.

Amylose starch consists of very few reducing ends compared to native starch. Therefore it is commercially important to identify genes that further enhance the amylose biosynthesis and that are capable to incorporate the excess of glucose residues available and to compensate the decrease in starch content in plants that produces amylose in high amounts.

The invention aims at enhancing the yield of amylose biosynthesis by the over-expression of genes which enhance starch biosynthesis in transformed plants.

The invention describes genes coding for proteins which enhance starch production.

The present invention describes the nucleic acids SEQ ID NO 1 and 3 from potato coding for enzymes enhancing the de novo starch biosynthesis.

Example 1 describes that the nucleic acid sequences SEQ ID NO 1 or 3 can complement a missing glycogenin function in yeast cells containing knock-out mutations for the self-glycosylating proteins Glg1p and Glg2p.

Gene constructs were made for gene-inhibition and over-expression of the two genes SEQ ID NO 1 or 3 in potato. Transgenic lines with the over-expressed or inhibited enzyme activity were analysed with regard to the genes influence on starch content.

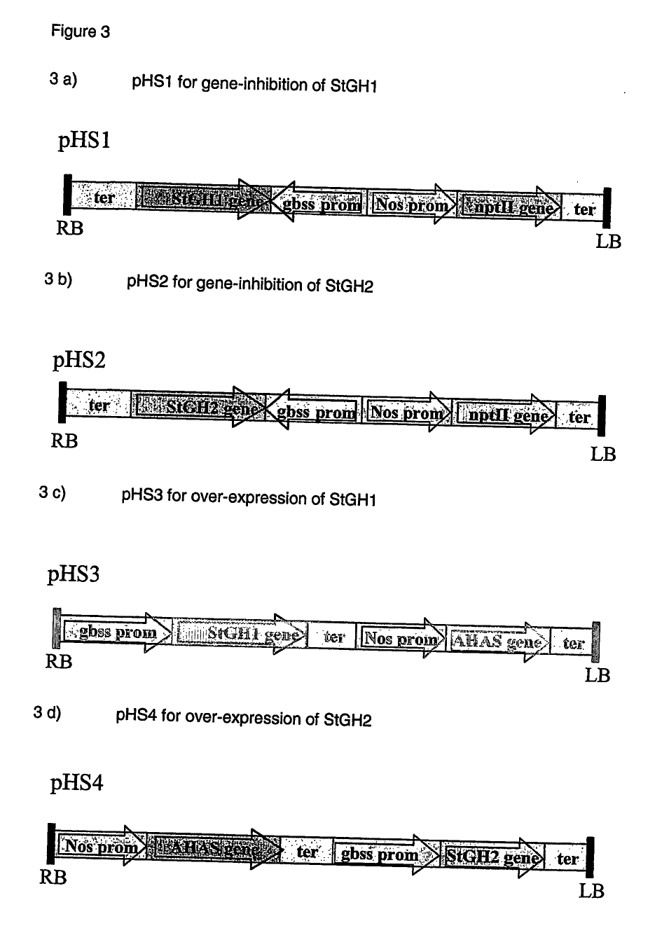

Both genes were inserted in sense and antisense direction downstream of a plant promoter element, resulting in the transformation binary vectors pHS1, pHS2, pHS3 and pHS4, see FIGS. 3-7.

The antisense constructs were transformed into the potato plant varieties Prevalent and Producent and the sense constructs were transformed to the potato variety Desiree and the transgenic plant AM99-2003 according to the transformation method as described in example 2. The transgenic plant AM99-2003 was produced as described in example 3.

Prevalent and Producent are starch varieties having a starch content of approximately 20%. Desiree is a potato variety having a starch content of approximately 16% and AM99-2003 is a transgenic high amylose line having a starch and thereby amylose content of approximately 13%.

The putative genes were isolated from a tuber specific cDNA library of Solanum tuberosum (variety Prevalent). The library was made from a lambdaZAP directional kit (Stratagene).

Both cDNAs isolated were full-length clones of the individual genes and named StGH1 and StGH2, for nucleic acid sequences see SEQ ID NO 1 and SEQ ID NO 3.

pHS1

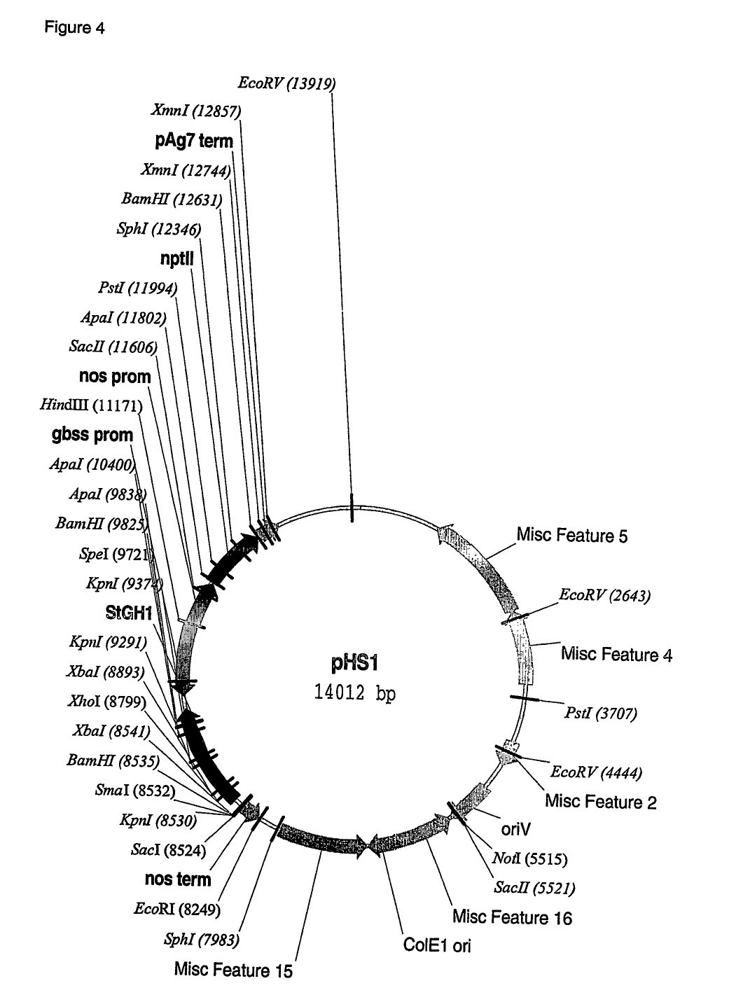

A 1300 bp PCR fragment from the StGH1 gene was constructed in antisense direction driven by the gbss promoter. The PCR fragment was cut out from its cloning vector pCR4-TOPO (Invitrogen) with EcoRI (blunted) and XbaI. The fragment was ligated to the pGPTV-kan (Becker, D. et al., Plant Molecular Biology 20:1195-1197(1992 ) based binary vector pHo3.1 between a gbss promoter (WO 92/11376) and a nos terminator at the SalI (blunted) and XbaI sites. The binary vector also includes nptII as selection marker driven by the nos promoter (Herrera, L. et al., 1983). The construct was named pHS1, for details see FIG. 3a and 4.

pHS2

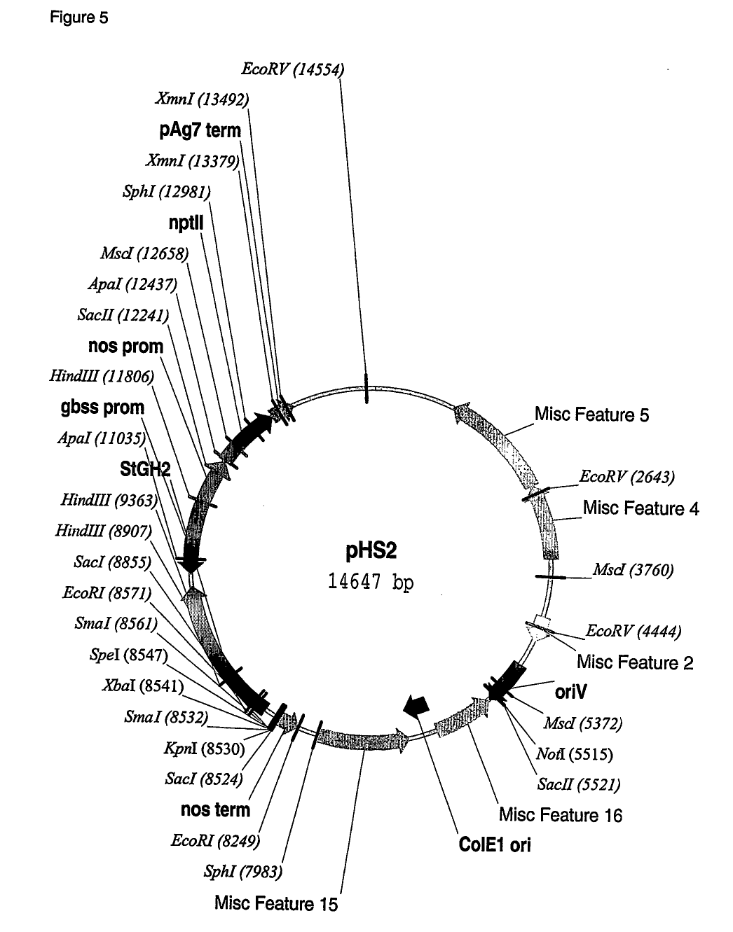

A 2300 bp full-length cDNA clone of StGH2 was cut out from the cloning vector pBluescript (Stratagene) with XbaI and XhoI. The gene was ligated in antisense direction between the gbss promoter and nos terminator to the binary vector pHo3.1 at XbaI and SalI. As can be seen under pHS1 the vector has nptII as selection system. The vector was named pHS2, for details see FIGS. 3b and 5.

pHS3

A full-length StGH1 cDNA, (1780 bp) was cut out from the host vector pBluescript with EcoRI (blunted) and BglII and ligated to the BamHI and SmaI sites of pUCgbssprom (3886 bp), containing pUC19 with the gbss promoter and the nos terminator. The plasmid was named pUCGH1.

A fragment with the gbss promoter, the StGH1 gene and the nos terminator was moved from pUCGH1 with EcoRI (blunted) and HindIII (2980 bp) and ligated to PstI (blunted) and HindIII opened pSUN1 (WO 02/00900). The plasmid was named pSUNGH1.

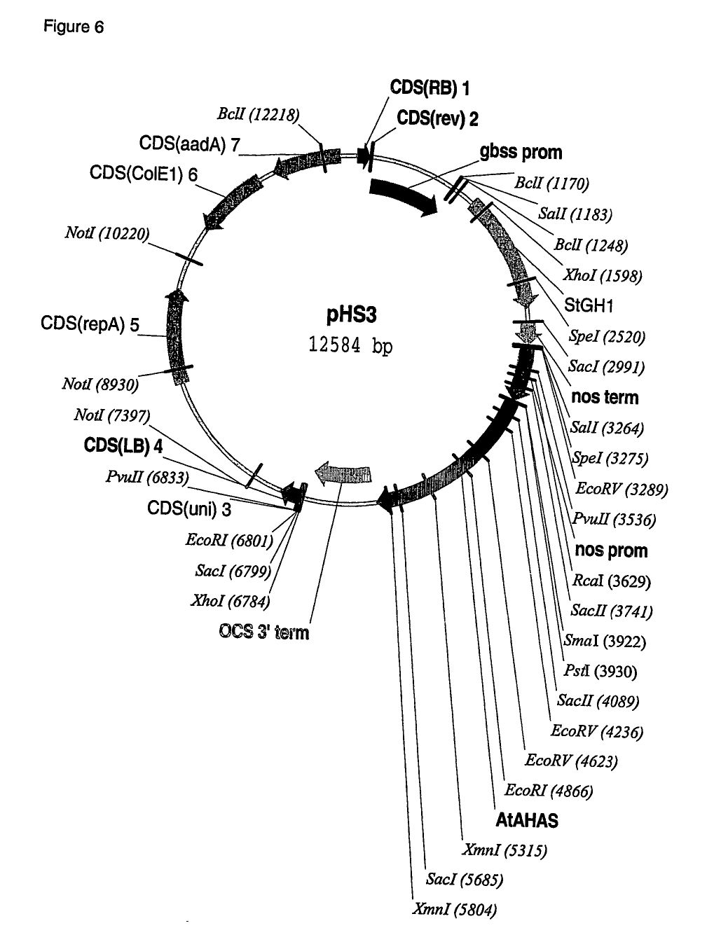

A 3600 bp fragment containing the AHAS resistance gene from Arabidopsis thaliana (Sathasivan, K. et al., Plant Physiology 97(1991), 1044-1050) with nos promoter, see Herrera-Estrella, L. et al., Nature 303:209-213(1983) and OCS terminator (Wesley, S. V. et al., Plant J. 27(6):581-590(2001) was ligated to pSUNGH1 (9000 bp) at the SmaI site. The vector was given the name pHS3, for details see FIGS. 3c and 6.

pHS4

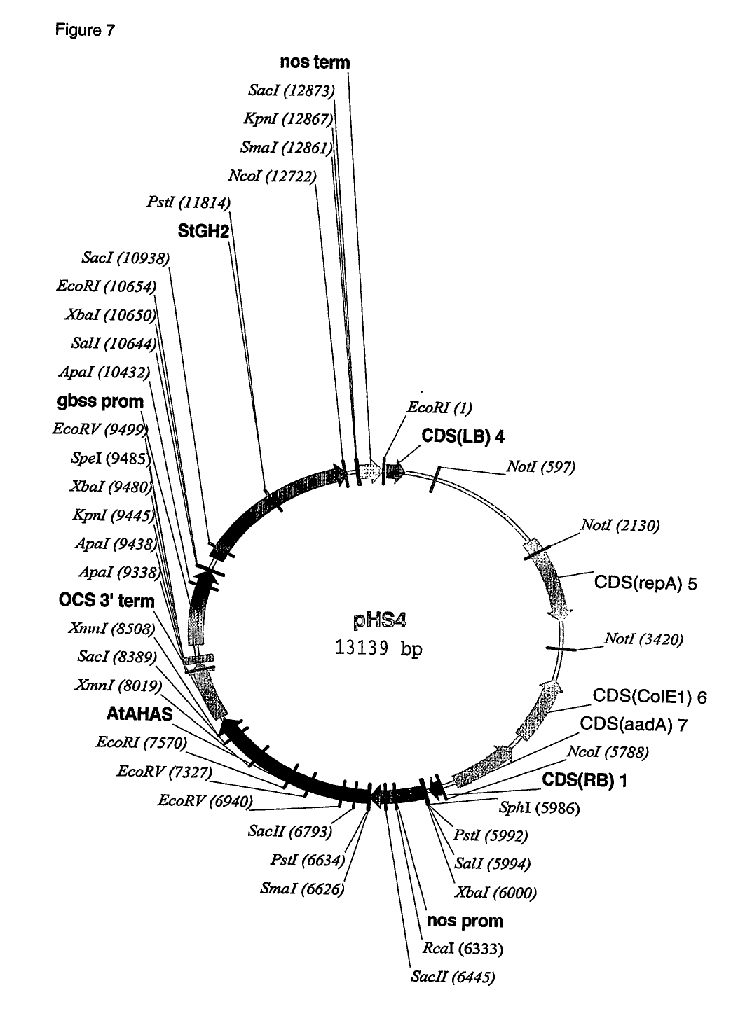

The gbss promoter and nos terminator was ligated to pBR322 with EcoRI and HindIII. Between the promoter and terminator an EcoRI-HincII full-length gene pStGH2 was cloned at the XbaI site. The 3366 bp promotor-gene-terminator complex was cut using EcoRI (partial digestion) and EcoRV, and ligated to pSUN1 at EcoRI-EcoRV and named pSUNGH2. An XbaI fragment with AHAS gene (Arabidopsis thaliana), nos promoter and OCS terminator was ligated to pSUNGH2 opened with XbaI (partial digestion). The AHAS gene is used as selection marker. The construct was named pHS4, for details see FIGS. 3d and 7.

Example 2 describes the general method for the transformation of different potato plant varieties producing native starch or high amylose type starch with pHS1, pHS2, pHS3 or pHS4.

The StGH1 and StGH2 genes were down-regulated in the potato plant varieties Prevalent and Producent by transformation with the genes in antisense direction in relation to a plant regulatory element as described in example 4 and 6. Down-regulation of the two genes resulted in a decrease in gene expression in transgenic lines compared to their mother varieties in the order of 50-95%, see example 7 and table 3. Transgenic lines transformed with pHS1 and pHS2 with confirmed decrease in gene-expression have a decrease in dry matter of 7 to 11% compared to their mother varieties, see example 8 and table 5.

The StGH1 and StGH2 genes were over-expressed in potato driven by the tuber specific promoter gbss, as described in example 5. A mutated AHAS gene was used as selection marker yielding tolerance to the Imazamox herbicides. Two potato varieties were transformed, Desiree and AM99-2003 a transgenic high amylose line with a 40% decrease in starch content compared to its parental line. The transformed lines over-expressing StGH1 and StGH2 were selected as described in example 6. The gene expression levels were analysed with real-time PCR, see example 7 and table 3. The over-expression of the genes StGH1 and StGH2 resulted in a 2 to 10 times increase in gene expression compared to their parental line. Furthermore the lines over-expressing StGH1 and StGH2 showed an increase in dry matter of up to 36% as described in example 8 and table 5.

The over-expression of StGH1 and StGH2 in transgenic potato plants producing amylose type starch resulted in an increased dry matter content, which means an increased amylose content as no amylopectin is produced, see examples 8 to 12.

RNA interference (RNAi) functions by introduction of double stranded RNA (dsRNA) into a cell, which causes a degradation of the homologous RNA. The dsRNA is cleaved into small interfering RNA (siRNA) of 21-25 nucleotides by a ribonuclease called Dicer. The siRNA connects with a protein complex and forms a RNA-induced silencing complex RISC. The RISC becomes activated by ATP generated unwinding of the siRNA, which binds to the homologous transcript and cleaves the mRNA resulting in gene silencing, see Mc Manus M T and Sharp P A., Gene silencing in mammals by small interfering RNAs. Nature Rev Genet 3:737-747(2002);

Dillin A., The specifics of small interfering RNA specificity. Proc Natl Acad Sci USA 100(11):6289-6291 (2003);

Tuschl T., Expanding small RNA interference. Nature Biotechnol 20:446-448 (2002)

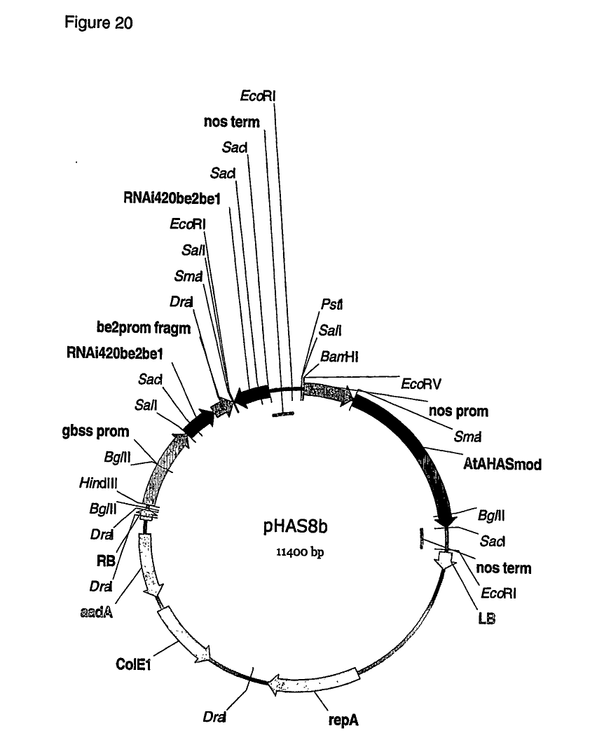

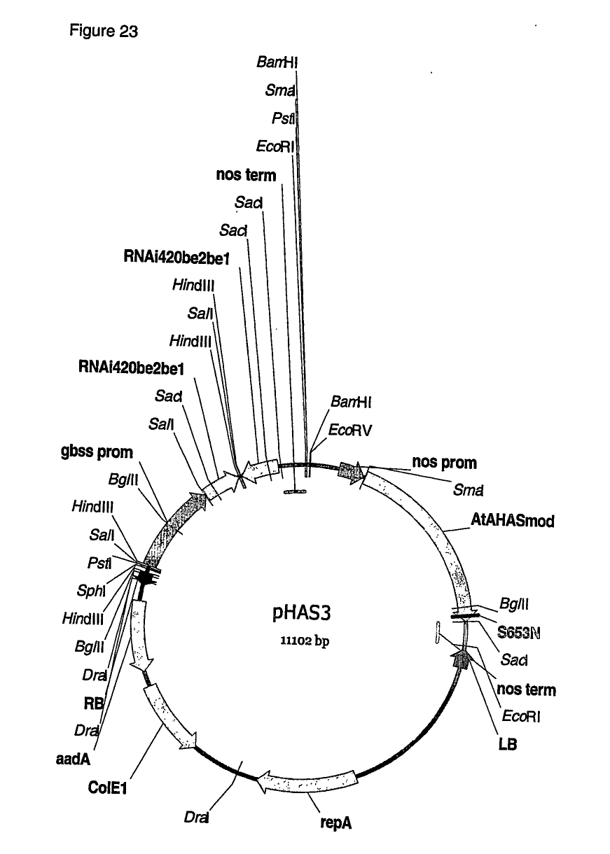

Production of high amylose lines was more efficient when using the RNAi constructs pHAS3 (FIG. 23) and pHAS8b (FIG. 20) than the antisense construct pHAbe12A. The frequency of high amylose lines of total transgenic shoots produced when using for example pHAS8b and pHAS3 is above >25%, compared to a frequency of approximately 1% high amylose lines of total transgenic shoots produced, see example 15 to 17.

The RNAi constructs pHAS8b (FIG. 20) and pHAS3 (FIG. 23) (SEQ ID NO 24) used for high amylose potato production contain a be1 and be2 fragment (SEQ ID NO 19) cloned in inverted tandem. The constructs are only differing in the spacer used located between the inverted repeats where for pHAS8b a fragment of the be2 promoter was used (SEQ ID NO 18) while for pHAS3 a cloning residue from pBluescript was used (SEQ ID NO 23). The RNAi constructs resulted in efficient down-regulation of the branching enzyme genes.

Furthermore, the fragments of respective be1 and be2 genes could be shorter or longer and could be targeting other parts of the branching enzyme genes. Shorter fragments for RNAi of be1 and be2 are described in SEQ ID NO 21 and 22.

The starch biosynthesis enhancing protein according to the invention comprises the amino acid sequence SEQ ID NO 2 or 4 or a protein which comprises a sequence derived from SEQ ID NO 2 or 4, which is at least 50%, preferably at least 60%, more preferably at least 70%, more preferably at least 80%, still more preferably at least 90%, most preferably at least 95%, identical at the amino acid level to the sequence SEQ ID NO 2 or 4 and has the property of a starch biosynthesis enhancing protein. This starch biosynthesis enhancing protein may also be prepared by artificial variations starting from the SEQ ID NO 2 or 4, for example by substitution, insertion or deletion of amino acids.

Such a protein can be used to increase the production of amylose or amylopectin in non-transgenic or transgenic plants.

The term “substitution” in the specification means the replacement of one or more amino acids by one or more amino acids. Preference is given to carrying out “conservative” replacements in which the amino acids replaced has a property similar to that of the original amino acid, for example replacement of Glu by Asp, Gin by Asn, Val by Ile, Leu by Ile, Ser by Thr.

“Deletion” is the replacement of an amino acid or amino acids by a direct bond. Preferred positions for deletions are the polypeptide termini and the junctions between the individual protein domains.

“Insertions” are insertions of amino acids into the polypeptide chain, with a direct bond formally being replaced by one or more amino acids.

“Identity” between two proteins means the identity of the amino acids over the in each case entire length of the protein, in particular the identity which is calculated by comparison with the aid of the Vector NTI Suite 7.1 Software of the company Informax (USA) using the Clustal W method (Thompson, J D et al., Nucleic Acid Research, 22 (22):4673-4680, 1994)

with the parameters set as follows:

| Multiple alignment parameter: | |
|---|---|
| Gap opening penalty | 15 |
| Gap extension penalty | 6.66 |
| Gap separation penalty range | 8 |
| Gap separation penalty | on |
| % identity for alignment delay | 40 |
| Residue specific gaps | on |
| Hydrophilic residue gap | off |
| Transition weighing | 0 |
| Pairwise alignment parameter: | |
| FAST algorithm | off |
| K-tuple size | 2 |
| Gap penalty | 5 |
| Window size | 4 |
| Number of best diagonals | 4 |

Accordingly, a protein which is at least 50% identical at the amino acid level to the sequence SEQ ID NO 2 or 4 means a protein which, when comparing its sequence with the sequence SEQ ID NO 2 or 4, is at least 50% identical, in particular according to the above program algorithm using the above set of parameters.

Further natural examples of genes coding for a starch biosynthesis enhancing protein according to the invention can readily be found, for example, in various organisms, in particular in plants, whose genomic sequence is known by comparing the identity of the amino acid sequences or of the corresponding back-translated nucleic acid sequences from databases with the sequence of SEQ ID NO 2 or 4, in particular according to the above program algorithm using the above set of parameters.

In the completed genome sequence of Arabidopsis thaliana, five putitative coding sequences can be deduced by searching for exon/intron boundaries and comparing with back translated sequences of SEQ ID NO 2 or 4.

The following nucleic acid sequences of Arabidopsis thaliana SEQ ID NO 5, SEQ ID NO 7, SEQ ID NO 9, SEQ ID NO 11 and SEQ ID NO 13 could be used to carry out the invention and are coding for the starch biosynthesis enhancing proteins SEQ ID NO 6, SEQ ID NO 8, SEQ ID NO 10, SEQ ID NO 12 and SEQ ID NO 14.

Furthermore the following nucleic acid sequences or ESTs can be used in order to identify and clone genes coding for a starch biosynthesis enhancing protein from plant organisms:

Tomato ESTs from GenBank: AW216407, BE450055, BF097262, BE450557, BF097173

Wheat ESTs from GenBank: BJ292476, BJ278875, BJ283925, BE442966, CA666180, BQ483228

Maize EST from GenBank: BG319971

Rice ESTs from GenBank: AL606633, CA752890, B1813265

Natural examples of starch biosynthesis enhancing proteins and the corresponding genes can furthermore readily be found in various organisms, in particular plants, whose genomic sequence is unknown by hybridization techniques in a manner known per se, for example starting from the nucleic acid sequences SEQ ID NO 1 or SEQ ID NO 3 or any of the SEQ ID NO 5, 7, 9, 11 or 13 or any of the EST sequences described above.

The hybridization may be carried out under moderate (low stringency) or, preferably, under stringent (high stringency) conditions.

Such hybridization conditions are described, inter alia, in Sambrook, J., Fritsch, E. F., Maniatis, T., in: Molecular Cloning (A Laboratory Manual), 2^{nd} edition, Cold Spring Harbor Laboratory Press, 1989, pages 9.31-9.57 or in Current Protocols in Molecular Biology, John Wiley & Sons, N.Y. (1989), 6.3.1-6.3.6.

By way of example, the conditions during the washing step may be selected from the range of conditions which is limited by those with low stringency (with 2×SSC at 50° C.) and those with high stringency (with 0.2×SSC at 50° C., preferably at 65° C.) (20×SSC: 0.3 M sodium citrate, 3 M sodium chloride, pH 7.0).

In addition, the temperature may be raised during the washing step from moderate conditions at room temperature, 22° C., to stringent conditions at 65° C.

Both parameters, salt concentration and temperature, may be varied simultaneously and it is also possible to keep one of the two parameters constant and to vary only the other one. It is also possible to use denaturing agents such as, for example, formamide or SDS during hybridization. In the presence of 50% formamide, the hybridization is preferably carried out at 42° C.

Some exemplary conditions for hybridization and washing step are listed below:

(1) hybridization conditions with, for example
(i) 4×SSC at 65° C., or
(ii) 6×SSC at 45° C., or
(iii) 6×SSC at 68° C., 100 mg/ml denatured fish sperm DNA, or
(iv) 6×SSC, 0.5% SDS, 100 mg/ml denatured fragmented salmon sperm DNA at 68° C., or
(v) 6×SSC, 0.5% SDS, 100 mg/ml denatured fragmented salmon sperm DNA, 50% formamide at 42° C., or
(vi) 50% formamide, 4×SSC at 42° C., or
(vii) 50% (vol/vol) formamide, 0.1% bovine serum albumin, 0.1% FicoII, 0.1% polyvinylpyrrolidone, 50 mM sodium phosphate buffer pH 6.5, 750 mM NaCl, 75 mM sodium citrate at 42° C., or
(viii) 2× or 4×SSC at 50° C. (moderate conditions), or
(ix) 30 to 40% formamide, 2× or 4×SSC at 42° C. (moderate conditions).

(2) Washing steps of 10 minutes each with, for example
(i) 0.015 M NaCl/0.0015 M sodium citrate/0.1% SDS at 50° C., or
(ii) 0.1×SSC at 65° C., or
(iii) 0.1×SSC, 0.5% SDS at 68° C., or
(iv) 0.1×SSC, 0.5% SDS, 50% formamide at 42° C., or
(v) 0.2×SSC, 0.1% SDS at 42° C., or
(vi) 2×SSC at 65° C. (moderate conditions).

Preferred proteins with starch biosynthesis enhancing activity are proteins from plants, cyanobacteria, mosses or algae, particular preferred from plants. A particular preferred protein comprises the amino acid sequence SEQ ID NO 2 or 4.

If, for example, the protein is to be expressed in a plant, it is frequently advantageous to use the codon usage of said plant for backtranslation and resynthesis of the gene according to codon usage of said plant.

The invention further relates to nucleic acids encoding a starch biosynthesis enhancing protein according to the invention. All of the nucleic acids mentioned in the specification may be, for example, a RNA sequence, DNA sequence or cDNA sequence.

Suitable nucleic acid sequences can be obtained, for example, by back-translating the polypeptide sequence according to the genetic code. For this, preference is given to using those codons which are used frequently according to the organism-specific codon usage. The codon usage can be readily determined on the basis of computer analyses of other known genes of the organisms in question.

All of the above-mentioned genes coding for a starch biosynthesis enhancing protein can furthermore be prepared in a manner known per se from the nucleotide building blocks by chemical synthesis, for example by fragment condensation of individual overlapping complementary nucleic acid building blocks of the double helix. The chemical synthesis of oligonucleotides may be carried out, for example, in a known manner according to the phosphoramidite method (Voet, Voet, 2nd edition, Wiley Press New York, pages 896-897). The annealing of synthetic oligonucleotides and filling-in of gaps with the aid of the Klenow fragment of DNA polymerase and ligation reactions and also general cloning methods are described in Sambrook et al. (1989), Molecular cloning: A laboratory manual, Cold Spring Harbor Laboratory Press.

Genes coding for this function may be integrated in the plant chromosomes and upon expression utilize a transit peptide to localise to plastids which is the organelle where starch/amylose biosynthesis takes place or be integrated directly into the plastid genome and thereby surpass the need for the localisation signal. The genes may be expressed constitutively or organ specific. For organ specific expression, promoters with tuber specific expression is preferable in potatoes while in cereals as maize or wheat a endosperm specific expression would be preferred to achieve a high degree of expression in organs where storage starch is accumulated. When transformed to the plastid genome then specific regulatory elements suitable for that organelle apply.

The genes of this invention may be used in combination with other genes that can be situated on the same gene construct or transferred and combined by co-transformation or super transformation. Genes and traits that are of interest to combine with the genes of the instant invention are agronomic or input trait such as herbicide tolerance, disease and pest resistance or stress tolerance but could also be output traits such as starch structure modification or yield. Genes and traits used in combination with the genes described in the invention could be for adding a function that is not present in the modified plant species or over-expressing a function that is already present or inhibiting a function by the use of antisense, RNAi or antibodies.

The invention may be used to increase the starch or amylose content in potato tubers but would in its context not be limited to potatoes but would be applicable to other starch producing and storing plants such as e.g. corn, cassava, wheat, barley, oat and rice.

The described invention is particularly suited for eliminating a lower starch content associated with increased amylose content in different plants where the number of α-1,4-glucan chain non-reducing ends is greatly reduced due to the reduction or elimination of α-1,6 branch formation. Amylopectin is an extremely efficient structure, as is glycogen, for polysaccharide production since it is very branched and thus contains as many points accessible for starch synthesis as there are non-reducing ends. Starch that is mainly composed of amylose, contains much fewer branches and thus the biosynthetic capacity is reduced. In order to enhance starch biosynthesis when there is no amylopectin production, expression of genes as described in the present invention, could for example form new primers that can replace amylopectin as a source for starch biosynthesis capacity and thereby reduce or eliminate the lost capacity for starch synthesis. To further illustrate the situation the degree of branching in ordinary potato starch is approximately 3.1% while in high amylose starch it is 0.3-1.0% depending on amylose content. This decrease of branching and starch content is further associated with an increase in glucose and fructose content.

The increased amylose content and thereby solids content is also advantageous for the processing properties in various applications such as for french fries, potato crisps and other potato based products. In addition to an increased solid content, the inserted genes SEQ ID NO 1 or 3 of the present invention result in the transformation of excess sugars into α-1,4-glucan chains and thereby reducing browning of fried potato products, Maillard reaction, in which amino acids react with free sugars.

Furthermore
(i) any gene of plant origin with the described activity can be used for increasing amylose content and solids
(ii) the genes can be controlled by any regulating promoter element functional in plant.
(iii) any starch producing crop of any variety can be transformed with the described genes.
(iiii) any plant transformation method can be used.
(iiiii) any binary vector can be used for the insertion of the described genes.
(iiiiii) the described genes can be combined with any other desired transgenically inserted traits.

The invention further relates to a method for producing amylose by culturing plants which have, compared to a wild type or a genetically modified plant producing already amylose type starch, an increased amylose biosynthesis activity, said proteins comprising the amino acid sequence SEQ ID NO 2 or 4 or a sequence which is derived from one of these sequences by substitution, insertion or deletion of amino acids and which is at least 50% identical at the amino acid level to the sequence SEQ ID NO 2 or 4.

Increased amylose biosynthesis activity compared to the wild type or transgenic line means that the amount of amylose formed is increased by the starch biosynthesis enhancing protein in comparison with the wild type or transgenic line.

This increase in starch or amylose biosynthesis activity is preferably at least 5%, further preferably at least 10%, further preferably at least 20%, further preferably at least 50%, more preferably at least 100%, still more preferably at least 200%, in particular at least 500%, of the protein activity of the wild type or transgenic line.

A “wild type” means the corresponding genetically unmodified starting plant. This plant is preferably Solanum tuberosum.

Depending on the context, the term “plant” means a wild type starting plant or a genetically modified starting plant.

“Transgenic plant” or “genetically modified plant” means that the plant contains an additional inserted gene segment that may be foreign or endogenous to the plant species, additional genes or additional gene fragments in sense and/or antisense orientation to a suitable promoter corresponding to the following polypeptides and showing enzymatic activity of a starch branching enzyme I, a starch branching enzyme II and/or the starch biosynthesis enhancing protein as specified in SEQ ID NO 1 or 3 or polynucleotides having at least 60% sequence identity thereof.

“Amylose type starch” means that the amylose content of the starch is increased compared to the amylose content of starch produced by wild type plants especially wild type potato plants.

The starch or amylose biosynthesis activity may be increased in various ways, for example by eliminating inhibiting regulatory mechanisms at the translation and protein levels or by increasing the gene expression of a nucleic acid encoding a starch biosynthesis enhancing protein compared to the wild type or transgenic plant, for example by inducing a gene encoding the starch biosynthesis enhancing protein via activators or by introducing into the plant nucleic acids encoding a starch biosynthesis enhancing protein.

According to the invention, increasing the gene expression of a nucleic acid encoding a starch biosynthesis enhancing protein could also mean manipulating the expression of the endogenous starch biosynthesis enhancing protein intrinsic to the plant, in particular in potato plants. This may be achieved, for example, by modifying the promoter DNA sequence of genes encoding a starch biosynthesis enhancing protein. Such a modification which leads to a modified or preferably increased rate of expression of at least one endogenous gene encoding a starch biosynthesis enhancing protein may be carried out by deleting or inserting DNA sequences.

It is also possible to modify expression of one or more endogenous starch biosynthesis enhancing protein by applying exogenous stimuli. This may be carried out by particular physiological conditions, i.e. by applying foreign substances.

Furthermore, it is possible to achieve a modified or increased expression of at least one endogenous gene encoding a starch biosynthesis enhancing protein by the interaction of a regulatory protein which is modified or is not present in the untransformed plant.

In a preferred embodiment, the starch biosynthesis enhancing protein activity is increased compared to the wild type or transgenic plant by increasing the gene expression of a nucleic acid encoding a starch biosynthesis enhancing protein, said starch biosynthesis enhancing protein comprising the amino acid sequence SEQ ID NO 2 or 4 or a sequence which is derived from said sequences by substitution, insertion or deletion of amino acids and which is at least 50% identical at the amino acid level to the sequence SEQ ID NO 2 or 4.

In the case of genomic nucleic acid sequences encoding a starch biosynthesis enhancing protein from eukaryotic sources, which contain introns, preferably already processed nucleic acid sequences such as the corresponding cDNAs are to be used, if the host organism is unable to or cannot be enabled to express the corresponding starch biosynthesis enhancing protein.

In this preferred embodiment, the transgenic plant of the invention thus contains, compared to the wild type or transgenic plant, at least one further gene encoding a starch biosynthesis enhancing protein. In this preferred embodiment, the genetically modified plant of the invention has accordingly at least one transgenic endogenous or exogenous nucleic acid encoding a starch biosynthesis enhancing protein.

Suitable and preferred nucleic acids are described above. In a particularly preferred embodiment, a nucleic acid comprising the sequence SEQ ID NO 1 or 3 is introduced into the plant.

According to the invention, organisms means preferably eukaryotic organisms, such as, for example, yeasts, algae, mosses, fungi or plants, which are capable of producing starch or amylose, either as wild type or enabled by genetic modification. Preferred organisms are photosynthetically active organisms such as, for example, plants which, even as a wild type, are capable of producing starch or amylose type starch.

Particularly preferred organisms are potato plants.

The present invention furthermore relates to the use of proteins comprising the amino acid sequence SEQ ID NO 2 or 4 or a sequence which is derived from this sequence by substitution, insertion or deletion of amino acids and which is at least 50% identical at the amino acid level to the sequence SEQ ID NO 2 or 4 and having starch biosynthesis enhancing activity.

The present invention further relates to the use of nucleic acids SEQ ID NO 1 or 3 or one of the SEQ ID NOS 5, 7, 9, 11 or 13 encoding proteins having a starch biosynthesis enhancing activity in plants.

The transgenic organisms, in particular plants, are preferably prepared by transforming the starting organisms, in particular plants, with a nucleic acid construct containing the above-described nucleic acid, encoding a starch biosynthesis enhancing protein which is functionally linked to one or more regulatory signals ensuring transcription and translation in said organisms.

These nucleic acid constructs in which the coding nucleic acid sequence is functionally linked to one or more regulatory signals ensuring transcription and translation in organisms, in particular in plants, are also referred to as expression cassettes herein below.

Accordingly, the invention further relates to nucleic acid constructs, in particular to nucleic acid constructs functioning as expression cassette, which comprise a nucleic acid encoding a starch biosynthesis enhancing protein which is functionally linked to one or more regulatory signals ensuring transcription and translation in organisms, in particular in plants.

The regulatory signals preferably comprise one or more promoters ensuring transcription and translation in organisms, in particular in plants.

The expression cassettes include regulatory signals, i.e. regulatory nucleic acid sequences, which control expression of the coding sequence in the host cell. According to a preferred embodiment, an expression cassette comprises upstream, i.e. at the 5′ end of the coding sequence, a promoter and downstream, i.e. at the 3′ end, a polyadenylation signal and, where appropriate, further regulatory elements which are operatively linked to the coding sequence for at least one of the above-described genes located in between. Operative linkage means the sequential arrangement of promoter, coding sequence, terminator and, where appropriate, further regulatory elements in such a way that each of the regulatory elements can properly carry out its function in the expression of the coding sequence.

When the organism used is a plant, the nucleic acid constructs and expression cassettes of the invention preferably contain a nucleic acid encoding a plastid transit peptide ensuring localisation in plastids.

The preferred nucleic acid constructs, expression cassettes and vectors for plants and methods for preparing transgenic plants and also the transgenic plants themselves are described in examples 2 to 6 below.

The sequences preferred for operative linkage, but not limited thereto, are targeting sequences for ensuring subcellular localisation to plastids such as amyloplasts or chloroplasts but could also mean in the apoplasts, in the vacuole, in the mitochondrion, in the endoplasmic reticulum (ER), in the nucleus, in elaioplasts or in other compartments and translation enhancers such as the tobacco mosaic virus 5′-leader sequence (Gailie et al., Nucl. Acids Res. 15 (1987), 8693-8711).

A suitable promoter of the expression cassette is in principle any promoter which is able to control the expression of foreign genes in plants.

“Constitutive” promoter means those promoters which ensure expression in numerous, preferably all, tissues over a relatively long period of plant development, preferably during the entire plant development.

Preference is given to using, in particular, a promoter from plants or a promoter originating from a plant virus. Preference is in particular given to the promoter of the 35S transcript of the CaMV cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or the 19S CaMV promoter (U.S. Pat. No. 5,352,605; WO 84/02913; Benfey et al. (1989) EMBO J 8:2195-2202).

Another suitable constitutive promoter is the Rubisco small subunit (SSU) promoter (U.S. Pat. No. 4,962,028), the leguminB promoter (GenBank Acc. No. X03677), the Agrobacterium nopaline synthase promoter, the TR double promoter, the agrobacterium OCS (octopine synthase) promoter, the ubiquitin promoter (Holtorf S et al. (1995) Plant Mol Biol 29:637-649), the ubiquitin 1 promoter (Christensen et al. (1992) Plant Mol Biol 20 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits or the promoter of a proline-rich wheat protein (WO 91/13991), the Pnit promoter (Y07648.L, Hillebrand et al. (1998), Plant. Mol. Biol. 36, 89-99, Hillebrand et al. (1996), Gene, 170, 197-200) and other promoters of genes whose constitutive expression in plants is known to the skilled worker.

The expression cassettes may also contain a chemically inducible promoter (review: Gatz et al. (1997) Annu Rev Plant Physiol Plant Mol Biol 48:89-108) which may be used to control expression of the starch biosynthesis enhancing protein gene in the plants at a particular time. Promoters of this kind, such as, for example, the PRP1 promoter (Ward et al. (1993) Plant Mol Biol 22:361-366), salicylic acid-inducible promoter (WO 95/19443), a benzenesulfonamide-inducible promoter (EP 0 388 186), a tetracycline-inducible promoter (Gatz et al. (1992) Plant J 2:397-404), an abscisic acid-inducible promoter (EP 0 335 528) and an ethanol- or cyclohexanone-inducible promoter (WO 93/21334), may likewise be used.

Further examples of suitable promoters are fruit ripening-specific promoters such as, for example, the fruit ripening-specific promoter from tomato (WO 94/21794, EP 409 625). Development-dependent promoters partly include the tissue-specific promoters, since individual tissues are naturally formed in a development-dependent manner.

Furthermore, preference is given in particular to those promoters which ensure expression in tissues or parts of the plant, in which, for example, biosynthesis of starch or amylose or of the precursors thereof takes place. Preference is given, for example, to promoters with specificities for leaves, stems, roots, seeds and tubers.

Seed-specific promoters are, for example, the phaseoline promoter (U.S. Pat. No. 5,504,200; Bustos M M et al. (1989) Plant Cell 1 (9):839-53), the promoter of the 2S albumin gene (Joseffson L G et al. (1987) J Biol Chem 262:12196-12201), the legumin promoter (Shirsat A et al. (1989) Mol Gen Genet 215(2): 326-331), the USP (unknown seed protein) promoter (Bäumlein H et al. (1991) Mol Gen Genet 225(3):459-67), the promoter of the napin gene (U.S. Pat. No. 5,608,152; Stalberg K et al. (1996) L Planta 199:515-519), the sucrose-binding protein promoter (WO 00/26388) and the legumin B4 promoter (LeB4; Bäumlein H et al. (1991) Mol Gen Genet 225: 121-128; Baeumlein et al. (1992) Plant Journal 2(2):233-9; Fiedler U et al. (1995) Biotechnology (NY) 13(10):1090f), the Arabidopsis oleosin promoter (WO 98/45461), the Brassica Bce4 promoter (WO 91/13980) and the vicillin promoter (Weschke et al. 1988, Biochem. Physiol. Pflanzen 183, 233-242; Bäumlein H et al. (1991) Mol Gen Genet 225(3):459-67).

Further suitable seed-specific promoters are those of the genes coding for high molecular weight glutenine (HMWG), gliadin, branching enzyme, ADP glucose pyrophosphatase (AGPase) and starch synthase. Preference is further given to promoters which allow seed-specific expression in monocotyledons such as e.g. corn, barley, wheat, rye, rice, etc. It is also possible to use advantageously the promoter of the Ipt2 or Ipt1 gene (WO 95/15389, WO95/23230) or the promotors described in WO 99/16890 (promoters of the hordein gene, the glutelin gene, the oryzin gene, the prolamin gene, the gliadin gene, the glutelin gene, the zein gene, the kasirin gene and the secalin gene).

Examples of tuber-, storage root- or root-specific promoters are the patatin promoter class I (B33), the potato cathepsin D inhibitor promoter and the potato granular bound starch synthase (GBSS) promoter as described in EP-A 0 921 191.

Examples of leaf-specific promoters are the cytosolic FBPase promoter from potato (WO 97/05900), the rubisco (ribulose-1,5-bisphosphate carboxylate) SSU (small sub-unit) promoter and the potato ST-LSI promoter (Stockhaus et al. (1989) EMBO J 8:2445-2451).

Further promoters suitable for expression in plants have been described (Rogers et al. (1987) Meth in Enzymol 153:253-277; Schardl et al. (1987) Gene 61:1-1 1; Berger et al. (1989) Proc Natl Acad Sci USA 86:8402-8406).

The site of starch and amylose biosynthesis in potato plants is the amyloplast. Therefore amyloplast-specific targeting and activity of the gene products of the inventive nucleic acids SEQ ID NO 1 or 3 encoding a starch biosynthesis enhancing protein is desirable.

The expression may also take place in a tissue-specific manner in all parts of the plant.

A further preferred embodiment therefore relates to a tuber-specific expression of the nucleic acids SEQ ID NO 1 or 3.

In addition, a constitutive expression of the gene encoding a starch biosynthesis enhancing protein is advantageous. On the other hand, however, an inducible expression of this gene may also be desirable.

An expression cassette is preferably prepared by fusing a suitable promoter to an above-described nucleic acid encoding a starch biosynthesis enhancing protein and, preferably, to a nucleic acid which has been inserted between promoter and nucleic acid sequence and which codes for an amyloplast-specific transit peptide and also to a polyadenylation signal according to familiar recombination and cloning techniques as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

Particular preference is given to inserted nucleic acid sequences which ensure targeting in the amyloplasts.

It is also possible to use an expression cassette in which the nucleic acid sequence encodes a starch biosynthesis enhancing protein fusion protein, one part of the fusion protein being a transit peptide which controls translocation of the polypeptide. Preference is given to amyloplast-specific transit peptides which, after translocation of starch biosynthesis enhancing protein into the amyloplasts, are enzymatically cleaved off the starch biosynthesis enhancing protein part.

Particular preference is given to the transit peptide which is derived from the Nicotiana tabacum plastid transketolase or from another transit peptide (e.g. the transit peptide of the rubisco small subunit or of ferredoxin NADP oxidoreductase and also of isopentenyl pyrophosphate isomerase-2) or from its functional equivalent.

Further examples of a plastid transit peptide are the transit peptide of the plastid isopentenyl pyrophosphate isomerase-2 (IPP-2) from Arabidopsis thaliana and the transit peptide of the ribulose bisphosphate carboxylase small subunit (rbcS) from pea (Guerineau, F, Woolston, S, Brooks, L, Mullineaux, P (1988) An expression cassette for targeting foreign proteins into the chloroplasts. Nucl. Acids Res. 16: 11380).

Plant genes of the invention which encode a plant starch biosynthesis enhancing protein may already contain the nucleic acid sequence which encodes a plastid transit peptide. In this case, a further transit peptide is not required. For example, the Solanum tuberosum sequences of the starch biosynthesis enhancing protein of the invention SEQ ID NO 1 or 3 contain already a transit peptide sequence.

The nucleic acids of the invention may be prepared synthetically or obtained naturally or comprise a mixture of synthetic and natural nucleic acid components and may also be composed of various heterologous gene sections of various organisms.

As described above, preference is given to synthetic nucleotide sequences with codons which are preferred by plants. These codons which are preferred by plants may be determined from codons which have the highest frequency in proteins and which are expressed in most of the interesting plant species.

When preparing an expression cassette, it is possible to manipulate various DNA fragments in order to obtain a nucleotide sequence which expediently can be read in the correct direction and is provided with a correct reading frame. The DNA fragments may be linked to one another by attaching adaptors or linkers to said fragments.

It is furthermore possible to use manipulations which provide appropriate restriction cleavage sites or which remove excess DNA or restriction cleavage sites. In those cases for which insertions, deletions or substitutions such as, for example, transitions and transversions are suitable, in vitro mutagenesis, primer repair, restriction or ligation can be used.

Preferred polyadenylation signals are polyadenylation signals functional in plants, exemplified by those which correspond essentially to T-DNA polyadenylation signals from Agrobacterium tumefaciens, in particular of the T-DNA gene 3 (octopine synthase) or OCS terminator, the complete sequence of the Ti plasmid pTiACH5 (Gielen et al., EMBO J. 3, 835-846(1984) or functional equivalents.

The invention further relates to the use of the nucleic acids SEQ ID NO 1 or 3 for increasing the starch or amylose content in plants, e.g. potato plants which, as wild type, are capable of producing starch or amylose, see examples 2 to 13.

The invention is not limited to the over-expression of the nucleic acid sequences SEQ ID NO 1 or SEQ ID NO 3 in plants especially potato plants.

The over-expression of both nucleic acid sequences SEQ ID NO 1 and 3 in a plant can be used for enhancing amylose biosynthesis, see examples 14-16. Constructs containing the nucleic acids SEQ ID NO 1 and SEQ ID NO 3 can also be used for increasing the starch content or the amylopectin content in plants. These constructs can be made on the same T-DNA driven by one promoter each. These constructs can also be made on the same T-DNA in tandem driven by the same promoter. These constructs can also be transformed using more than one construct, either at the same time (co-transformation) or in different transformation events.

The above-described proteins and nucleic acids may be used for producing starch or amylose in transgenic plants.

The transfer of foreign genes into the genome of an organism, in particular of a plant, is referred to as transformation.

For this purpose, methods known per se for transforming plants and regenerating plants from plant tissues or plant cells can be used, in particular in plants, for transient or stable transformation, e.g. as described in example 2.

Suitable methods for the transformation of plants are the protoplast transformation by polyethylene glycol-induced DNA uptake, the biolistic method using the gene gun—also known as particle bombardment method, electroporation, the incubation of dry embryos in a DNA-containing solution, microinjection and the above-described Agrobacterium-mediated gene transfer. Said methods are described, for example, in B. Jenes et al., Techniques for Gene Transfer, in: Transgenic Plants, Vol. 1, Engineering and Utiliation, edited by S. D. Kung and R. Wu, Academic Press (1993), 128-143 and in otrykus, Annu. Rev. Plant Physiol. Plant Molec. Biol. 42 (1991), 205-225).

The construct to be expressed is preferably cloned into a vector which is suitable for transforming Agrobacterium tumefaciens, for example pBin19 (Bevan et al., Nucl. Acids Res. 12 (1984), 8711) or preferably pSUN2 (WO 02/00900).

Accordingly, the invention furthermore relates to vectors containing the above-described nucleic acids, nucleic acid constructs or expression cassettes.

Agrobacteria which have been transformed with an expression cassette can be used in a known manner for the transformation of plants, for example by bathing injured leaves or leaf sections in an Agrobacterium solution and then culturing them in suitable media.

Apart from in plants, the expression cassette may also be used for transforming bacteria, in particular cyanobacteria, mosses, yeasts, filamentous fungi and algae.

Genetically modified plants, also referred to as transgenic plants herein below, are preferably prepared by cloning the fused expression cassette which expresses a starch biosynthesis enhancing protein into a vector, for example pBin19, which is suitable for transforming Agrobacterium tumefaciens.

Agrobacteria which have been transformed with such a vector may then be used in a known manner for the transformation of plants, in particular of crop plants, for example by bathing injured leaves or leaf sections in an Agrobacterium solution and then culturing them in suitable media.

The transformation of plants by Agrobacteria is described, inter alia, in F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants which contain a gene for expression of a nucleic acid encoding a starch biosynthesis enhancing protein, which has been integrated into the expression cassette, can be regenerated in a known manner from the transformed cells of the injured leaves or leaf sections.

A host plant is transformed with a nucleic acid SEQ ID NO 1 or 3 encoding a starch biosynthesis enhancing protein by incorporating an expression cassette as insertion into a recombinant vector whose vector DNA comprises additional functional regulatory signals, for example sequences for replication or integration. Suitable vectors are described inter alia, in Methods in Plant Molecular Biology and Biotechnology (CRC Press), chapter 6/7, pp. 71-119 (1993).

By way of example, the plant expression cassette may be incorporated into a derivative of the transformation vector pBin-19 with 35s promoter (Bevan, M., Nucleic Acids Research 12: 8711-8721 (1984).

Using the above-cited recombination and cloning techniques, it is possible to clone the expression cassettes into suitable vectors for maintenance and propagation of genetic material for example in E. coli. Suitable cloning vectors are, inter alia, pBR322, pUC series, M13 mp series, pBluescript and pACYC184. Particularly suitable are binary vectors which can replicate both in E. coli and in agrobacteria.

The invention therefore further relates to the use of the above-described nucleic acids or of the above-described nucleic acid constructs, in particular of the expression cassettes, for preparing genetically modified plants or for transforming plants, plant cells, plant tissues or parts of plants.

The use is preferably aimed at increasing the starch or amylose content of the plant, of the tubers or in other parts of the plant.

The use is most preferably aimed at increasing the starch or amylose content of wild-type or transgenic potato plants and especially the tubers of wild-type or transgenic potato plants.

Accordingly, the invention further relates to a method for preparing genetically modified plants by introducing an above-described nucleic acid or an above-described nucleic acid construct into the genome of the starting organism.

The invention further relates to the genetically modified organisms, the genetic modification increasing the activity of a starch biosynthesis enhancing protein compared to a wild type or transgenic plant and the starch biosynthesis enhancing protein comprising the amino acid sequence SEQ ID NO 2 or 4 or a sequence which is derived from this sequence by substitution, insertion or deletion of amino acids and which is at least 50% identical at the amino acid level to the sequence SEQ ID NO 2 or 4.

As illustrated above, the starch biosynthesis enhancing protein activity is increased compared to the wild type or transgenic plant preferably by increasing the gene expression of a nucleic acid encoding a starch biosynthesis enhancing protein.

In a further preferred embodiment, gene expression of a nucleic acid encoding a starch biosynthesis enhancing protein is increased, as illustrated above, by introducing nucleic acids encoding a starch biosynthesis enhancing protein into the organism and thus by over-expressing nucleic acids encoding a starch biosynthesis enhancing protein.

Such transgenic plants, their propagation material and their plant cells, plant tissues, plant parts or tubers are a further subject of the present invention.

Genetically modified plants of the invention, which have an increased starch or amylose content and which can be consumed by humans and animals, can also be used as food- or feedstuffs or as feed and food supplements, for example directly or after processing known per se. The genetically modified plants may furthermore be used for producing starch or amylose-containing extracts of said plant and/or for producing feed and food supplements.

The invention further relates to:
I. A polynucleotide that encodes a polypeptide of SEQ ID NO 1 or 3.
II. A polynucleotide comprising at least 30 contiguous bases of SEQ ID NO 1 or 3.
III. A polynucleotide having at least 60% sequence identity to SEQ ID NO 1 or 3, wherein the identity is based on the entire coding sequence.
IV. A polynucleotide having at least 60% sequence identity to SEQ ID NO 1 or 3, wherein the % sequence identity is based on the entire sequence.
V. A polynucleotide which selectively hybridizes, under stringent conditions and a wash in 2×SSC at 50° C., to a hybridization probe derivable from the poly-ucleotide sequence as set forth in SEQ ID NO 1 or 3, or from the genomic sequence.
VI. A polynucleotide complementary to a polynucleotide of V.
VII. The polynucleotide of I, wherein the starch or amylose biosynthesis enhancing polynucleotide is from Solanum tuberosum.
III. The polynucleotide of I encoding a polypeptide, which after over-expression in a plant cell increases the starch or amylose content.
X. The polynucleotide of I in antisense orientation, which after expression in a plant cell decreases the starch or amylose content.
X. A vector comprising at least one polynucleotide of I.
XI. An expression cassette comprising at least one polynucleotide of I operably linked to a promoter, wherein the polynucleotide is in sense or antisense orientation.
XII. A host cell which is introduced with at least one expression cassette of X.
XIII. The host cell of XI that is a plant cell.
XIV. A transgenic plant comprising at least one expression cassette of XI.
XV. The transgenic plant of XIII, wherein the plant is Solanum tuberosum.
XVI. A tuber from the transgenic plant of XIV.
XVII. An isolated protein comprising a member selected from the group consisting of:
   a) a polypeptide comprising at least 10 contiguous amino acids of SEQ ID NO 2 or 4,
   b) a polypeptide which is a plant starch biosynthesis enhancing protein,
   c) a polypeptide comprising at least 55% sequence identity to SEQ ID NO 2 or 4, wherein the sequence identity is based on the entire sequence and has at least one epitope in common with a starch biosynthesis enhancing protein.
   d) a polypeptide encoded by a polynucleotide selected from SEQ ID NO 1 or 3,
   e) a polypeptide of SEQ ID NO 2 or 4.
XVIII. The protein of XVII, wherein the polypeptide is catalytically active.
XIX. A ribonucleic acid sequence encoding the protein of XVIII.
XX. A method for modulating the level of starch biosynthesis enhancing protein in a plant, comprising:
   a) stably transforming a plant cell with a polynucleotide coding for a starch biosynthesis enhancing protein operably linked to a promoter, wherein the polynucleotide is in sense or antisense orientation
   b) growing the plant cell under plant growing conditions to produce a regenerated plant capable of expressing the polynucleotide for a time sufficient to modulate the level of starch biosynthesis enhancing protein in the plant.
XXI. The method of XX, wherein the polynucleotide coding for a starch biosynthesis enhancing protein is selected from SEQ-ID NO 1 or 3.
XXII. The method of XX, wherein the plant is Solanum tuberosum.
XXIII. The method of XX, wherein activity of the starch biosynthesis enhancing protein is increased.
XXIV. A method for modulating the level of starch or amylose in a plant, comprising:
   a) stably transforming a plant cell with a polynucleotide coding for a starch biosynthesis enhancing protein operably linked to a promoter, wherein the polynucleotide is in sense or anti-sense orientation,
   b) growing the plant cell under plant growing conditions to produce a regenerated plant capable of expressing the polynucleotide for a time sufficient to modulate level of starch or amylose in the plant.
XXV. A method for modulating the level of starch or amylose in a plant, comprising:
   a) stably transforming a plant cell with a polynucleotide encoding a starch biosynthesis enhancing protein operably linked to a promoter, wherein the polynucleotide is in sense or anti-sense orientation.
   b) growing the plant cell under plant growing conditions to produce a regenerated plant capable of expressing the polynucleotide for a time sufficient to modulate level of starch or amylose in the plant.
XXVI. The method of XXIV wherein the polynucleotide coding for a starch biosynthesis enhancing protein is selected from SEQ ID NO 1 or 3.

Some of the terms used further on in the specification are defined at this point.

“Enzymatic activity/activity assay”: the term enzymatic activity describes the ability of an enzyme to convert a substrate into a product. In this context, both the natural substrate of the enzyme and a synthetic modified analog of the natural substrate can be used. The enzymatic activity can be determined in what is known as an activity assay via the increase in the product, the decrease in the starting material, the decrease or increase in a specific cofactor, or a combination of at least two of the aforementioned parameters as a function of a defined period of time.

“Functional equivalents” in the present context describe nucleic acid sequences which hybridize under standard conditions with the nucleic acid sequence encoding the starch biosynthesis enhancing protein or portions of the nucleic acid sequence encoding the starch biosynthesis enhancing protein, and which are capable of bringing about the expression of an enzymatically active plant starch biosynthesis enhancing protein in a cell or an organism.

It is advantageous to use short oligonucleotides of a length between 10 to 50 bp, preferably 15-40 bp, for example of the conserved or other regions, which can be determined via comparisons with other related genes in a manner known to the skilled worker for the hybridization. Alternatively, it is also possible to use longer fragments of the nucleic acids according to the invention or the complete sequences for the hybridization. These standard conditions vary depending on the nucleic acid used, namely oligonucleotide, longer fragment or complete sequence, or depending on which type of nucleic acid, that is DNA or RNA, is being used for the hybridization. Thus, for example, the melting temperatures for DNA:DNA hybrids are approx. 10° C. lower than those of DNA:RNA hybrids of equal length. Suitable hybridization conditions are described above.

A functional equivalent is furthermore also understood as meaning, in particular, natural or artificial mutations of the relevant nucleic acid sequences of the plant starch biosynthesis enhancing protein and their homologs from other organisms which make possible the expression of the enzymatically active plant starch biosynthesis enhancing protein in a cell or an organism.

Thus, the scope of the present invention also extends to, for example, those nucleotide sequences which are obtained by modification of the nucleic acid sequence of a starch biosynthesis enhancing protein. The purpose of such a modification can be, for example, the insertion of further cleavage sites for restriction enzymes, the removal of excess DNA, or the addition of further sequences. Proteins which are encoded via said nucleic acid sequences should still maintain the desired functions, despite the deviating nucleic acid sequence.

The term functional equivalent may also refer to the protein encoded by the nucleic acid sequence in question. In this case, the term functional equivalent describes a protein whose amino acid sequence is up to a specific percentage identical with that of he starch biosynthesis enhancing protein.

Functional equivalents thus encompass naturally occurring variants of the sequences described herein, and also artificial, for example chemically synthesized, nucleic acid sequences adapted to the codon usage, or the amino acid sequences derived therefrom.

In general, it can be said that functional equivalents independently of the amino acid sequence in question (encoded by a corresponding nucleic acid sequence) have in each case the enzymatic activity of a starch biosynthesis enhancing protein.

“Reporter genes” encode readily quantifiable proteins. Using these genes, an assessment of transformation efficacy or of the site or time of expression can be made via growth, fluorescence, chemoluminescence, bioluminescence or resistance assay or via photometric measurement (intrinsic color) or enzyme activity. Very especially preferred in this context are reporter proteins (Schenborn E, Groskreutz D. Mol. Biotechnol. 1999; 13(1):29-44) such as the “green fluorescence protein” (GFP) (Gerdes H H and Kaether C, FEBS Lett. 1996; 389(1):44-47; Chui W L et al., Curr. Biol. 1996, 6:325-330; Leffel S M et al., Biotechniques. 23(5):912-8,1997), chloramphenicol acetyl transferase, a luciferase (Giacomin, Plant Sci. 1996,116:59-72; Scikantha, J. Bact. 1996, 178:121; Millar et al., Plant Mol. Biol. Rep. 1992 10:324-414), and luciferase genes, in general b-galactosidase or b-glucuronidase (Jefferson et al., EMBO J. 1987, 6, 3901-3907), the Ura3 gene, the Ilv2 gene, the 2-desoxyglucose-6-phosphate phosphatase gene, b-lactamase gene, the neomycin phosphotransferase gene, the hygromycin phosphotransferase gene, or the BASTA (=gluphosinate) resistance gene.

“Significant increase”: referring to the enzymatic activity, is understood as meaning the increase in the enzymatic activity of the enzyme incubated with a candidate compound in comparison with the activity of an enzyme not incubated with the candidate compound, which lies outside an error in measurement.

“Substrate”: Substrate is the compound which is recognized by the enzyme in its original function and which is converted into a product by means of a reaction catalyzed by the enzyme.

Preferably, the plant starch biosynthesis enhancing protein is encoded by a nucleic acid sequence comprising
a) a nucleic acid sequence shown in SEQ ID NO 1 or 3; or
b) a nucleic acid sequence which, owing to the degeneracy of the genetic code, can be deduced from the amino acid sequence shown in SEQ ID NO 2 or 4 by back translation; or
c) a nucleic acid sequence which, owing to the degeneracy of the genetic code, can be deduced from a functional equivalent of the amino acid sequence shown in SEQ ID NO 2 or 4, which has an identity with SEQ ID NO 2 or 4 of at least 50%, by back translation.

The functional equivalent of SEQ ID NO 2 or 4 set forth in c) has an identity of at least 50%, 51%, 52%, 53%, 54%, 55%, 56%, 57% preferably at least 58%, 59%, 60%, 61%, 62%, 63%, 64%, 65%, 66%, 67%, 68%, 69%, and 70% more preferably 71%, 72%, 73%, 74%, 75%, 76%, 77%, 78%, 79%, 80%, 81%, 82%, 83%, 84%, 85% most preferably at least 86%, 87%, 88%, 89%, 90%, 91%, 92%, 93%, 94%, 95%, 96%, 97%, 98%, 99% identity with the SEQ ID NO 2 or 4.

Potato varieties used for starch production as well as genotypes with a high amylose content are transformed with gene constructs as described and in example 17 for the over-expression of a starch biosynthesis enhancing protein. The over-expression of StGH1 or StGH2 in potato plants will result in an increased starch or amylose content of the transgenic plant compared to the starting plant. The increase of starch content in the transgenic lines can be seen in table 9. The lines also show an increased harvest weight when grown In the greenhouse (table 6) thus resulting in an increased starch yield.

### EXAMPLE 1

Complementation Study in Yeast

Yeast contains two self-glycosylating proteins, Glg1p and Glg2p, which yield primers for the initiation of glycogen synthesis. For glycogen synthesis to take place in yeast it is required that either gene is functional. Yeast strain CC9, contain knock-out mutations for both genes and is therefore a null mutant regarding this specific biosynthetic function and is therefore unable to produce glycogen (Cheng, C. et al., Molecular and Cellular Biology (1995), 6632-6640). CC9 was used as a basis for complementation experiments with the isolated potato genes in order to validate their function by restoring glycogen biosynthesis in the CC9 strain. The potato genes were cloned in a yeast plasmid, pRS41 4 (Stratagene), and expressed with various yeast controlling elements such as Gal1, Adh1 and Glg2p promoters. CC9 was transformed by the resulting plasmids using LiCl and electroporation (Multiporator, Eppendorf). Transformed yeast colonies growing on appropriate media plates were screened by immersing in iodine solution. Wild type yeast producing glycogen is stained red brown by iodine while the null mutant CC9 is not stained. CC9 expressing the potato genes, StGH1 and StGH2, will stain red brown, when the isolated genes complement a glycogenin function in yeast and thus carry the desired function.

### EXAMPLE 2

Transformation Method

Fully expanded leaves from in vitro propagated potato plants are diagonally cut in 2-4 pieces and precultivated on MC-plates for 2-3 days at 23-24° C.

Agrobacterium tumefaciens strain LBA4404 containing pHS1, pHS2, pHS3 pHS4, pHASHS2, pHASHS4, pHASHS5, pHASHS6, pHASHS7 or pHASHS8 are grown in YEB medium with 100 μg rifampicin and 25 μg/ml kanamycin over night on constant shaking (200 rpm) at 28° C.

The Agrobacterium culture is prepared for infection by dilution 1:20 with MS10 medium. The leaf explants are infected for 8-10 min in the bacterial solution and afterwards drained on filter paper for 5-20 seconds. The leaf segments are placed on the MS300 plates for 2 days co-cultivation under modest light at 23-24° C. At the end of co-cultiation the leaf segments are moved to M400 plates containing 400 g/l Claforan to suppress bacterial growth. After 4-5 days the explants are moved to selection medium MS400 supplemented with 400 g/l Claforan. For explants transformed with pHS1 and pHS2 50 μM kanamycin was included in the media and for explants transformed with pHS3, pHS4, pHASHS2, pHASHS4, pHASHS5, pHASHS6, pHASHS7 and pHASHS8 0.5 M Imazamox was added to the media.

Leaf segments are transferred to fresh MS 400 selection medium every fortnight. The regenerated putative transgenic shoots are collected and cultivated on MS30 plates with 200 g/l Claforan aiming at shoot elongation.

When the shoots are 3-5 cm long, 1-2 cm are cut off and grown on microtuber medium in the dark at 25° C. After 2-5 weeks microtubers are produced.

| MC plates | MS300 |
|---|---|
| MS300 plates with 1.5-2 ml | 4.4 g/l MS-medium |
| liquid MS100 medium and | 2 mg/l naphthyl acetic acid |
| covered with one sterile | 1 mg/l 6-benzyl amino pyridine |
| filter paper | 3% (w/v) sucrose |
| | pH 5.2 |
| MS10 | MS400 |
| 4.4 g/l MS-medium (murashige and | 4.4 g/l MS-medium |
| Skoog) | 2 g/l zeatine |
| 1% (w/v) sucrose | 0.01 mg/l naphthyl acetic acid |
| pH 5.8 | 0.1 mg/l gibberellic acid |
| | 10% (w/v) sucrose |
| | 400 mg/l claforan |
| | 0.5 μM Imazamox or |
| | 50 μM kanamycin |
| | pH .8 |
| MS30 | Microtuber medium |
| 4.4 g/l MS-medium | 4.4 g/l MS-medium |
| 3% (w/v) sucrose | 2.5 mg/l kinetin |
| pH 5.8 | 0.5 mg/l abscisic acid |
| | 8% sucrose |
| | 200 mg/claforan |
| MS100 | |
| 4.4 mg/l MS-medium | |
| 30/l sucrose | |
| 0.5 m/lg thiamin-HCl | |
| 0.5 mg/l pyridoxin-HCl | |
| 1 mg/l nicotinacid | |
| 0.5 mg/l kinetin | |
| 29.8 mg/l ferrous sulfate hepta hydrate | |
| 1 mg/l 2,4-Dichlorophenoxyacetic acid | |
| 2 g/l caseinhydrolysate | |
| pH 5.2 | |

### EXAMPLE 3

Transgenic Plant AM 99-2003

High amylose potato lines can be produced for example by using antisense, RNAi or antibody technology that target the two starch branching enzymes starch branching enzyme 1 (SBE1) and starch branching enzyme 2 (SBE2).

The high amylose potato line AM99-2003 is produced by inhibition of the starch branching enzyme activities in the parental line Dinamo. Transformation is made with a construct of SBE1 and SBE2 in antisense orientation driven by the gbss promoter.

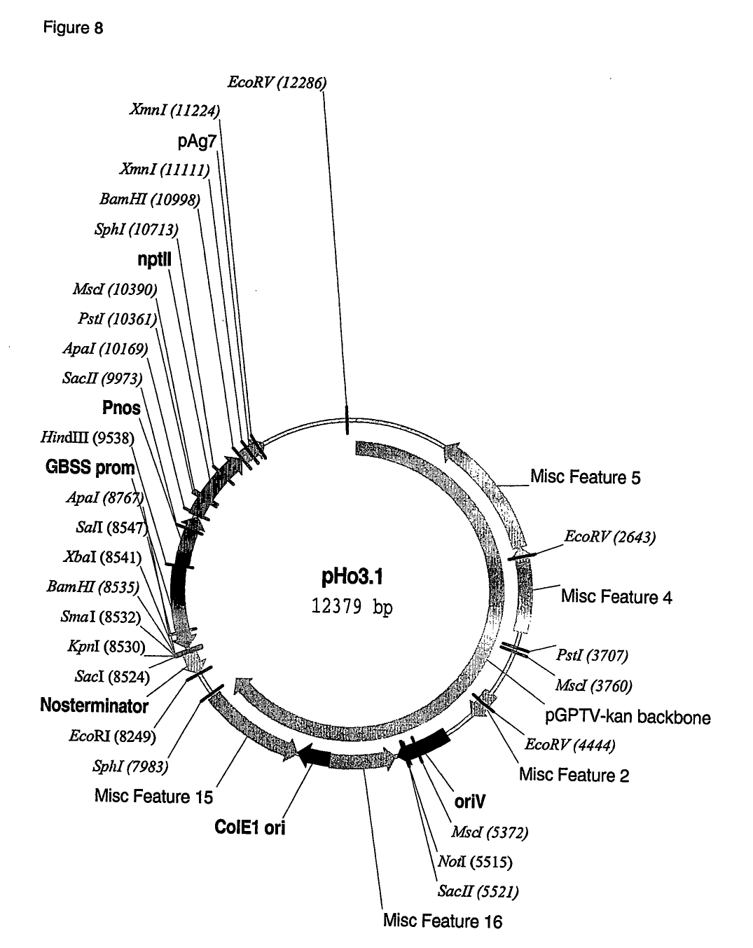

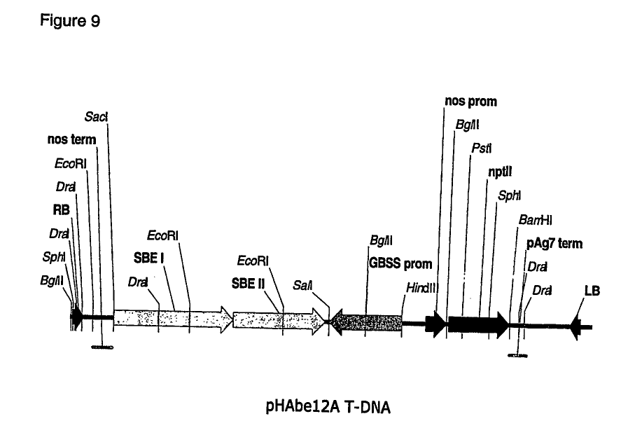

pBluescript containing a 1620 bp fragment of the 3′end of Sbe1 between EcoRV and SpeI is cut open with SpeI (blunt) and XbaI and ligated with a 1243 bp SstI (blunt) and XbaI fragment of the 3′end of Sbe2. The Sbe2 and Sbe1 complex is cut out with EcoRV and XbaI and ligated to the SmaI and XbaI opened up binary vector pHo3.1, see FIG. 8. The final vector is named pHAbe12A, see FIG. 9 and nucleic acid sequence SEQ ID NO 15. pHo3.1 is based on pGPTVKan (Becker, D. et al., Plant Molecular Biology 20 (1992), 1195-1197) with the addition of the 987 bp gbss promoter cloned at the HindIII site of pGPTVKan and the uidA gene is deleted by SmaI and SstI.

The parental line Dinamo is transformed with the construct pHAbe12A as described in example 2.

### EXAMPLE 4

Down-Regulation of StGH1 and StGH2 Genes in Potato by Antisense

The StGH1 and StGH2 genes were down-regulated in potato by transformation with the genes in antisense direction in relation to a plant regulatory element. The respective antisense genes were cloned in a binary vector driven by a tuber specific gbss promoter. NptII, yielding resistance to the antibiotic kanamycin, was used as selection marker. Two varieties were transformed, Prevalent and Producent. The shoots were selected on 50 μM kanamycin, which is a standard kanamycin concentration used for potato transformation (Ooms, G et al., Theoretical and Applied Genetics 73:744-750 (1987) and Tavazza, R. et al., Plant Science 59 (1988), 175-181).

### EXAMPLE 5

Over-Expression of StGH1 and StGH2 Genes in Potato

The StGH1 and StGH2 genes were over-expressed in potato driven by the tuber specific promoter gbss. A mutated AHAS gene was used as selection marker yielding tolerance to the Imazamox herbicides. Two potato varieties were transformed, Desiree and AM99-2003 a transgenic high amylose line with a 40% decrease in starch content compared to its parental line.

### EXAMPLE 6

Selection of Transgenic Lines

Non-transgenic escapes were identified and discarded by a PCR screening method. DNA was extracted according to DNeasy 96 Plant protocol (Qiagen). In a 96 well microtiter plate, 10-15 mg leaf tissue was added to each well together with a 5 mm steel ball, each well then representing one individual shoot. The plates were frozen in N₂(I) before homogenisation. The homogenisation was done at 30 Hz in a Mixermill300 for 1 min. The DNA was at the end of the extraction protocol eluted in 75 μl H₂O.

Specific primers for nptII and AHAS were used for the amplification of a 246 bp fragment respective a fragment of 509 bp for selection of successfully transformed lines.

| | |
|---|---|
| Npt2_for | 5′-AGCAAGGTGAGATGACAGGAGATC-3′ |
| Npt2_rev | 5′CAGACAATCGGCTGCTCTGATG-3′ |
| AHAS1_frw: | 5′-AACAACAACATCTTCTTOGATC-3′ |
| AHAS1_rev: | 5′-TAACGAGATTTGTAGCTCCG-3′. |

The PCR reactions were with the extracted DNA setup and run as follow:

Reaction:

| | |
|---|---|
| 10x PCR Mix | 2.0 μl |
| Primer frw (25 μM) | 0.4 μl |
| Primer rev (25 μM) | 0.4 μl |
| dNTPs (10 mM) | 0.4 μl |
| RedTAQ (Sigma) | 1.0 μl |
| Templat (˜20 ng/μl) | 4.0 μl |
| H₂O | 11.8 μl |

PCR program:

| | | |
|---|---|---|
| 94° C. | 30 s | |
| 59° C. | 30 s | x29 cycles |
| 72° C. | 30 s | |
| 72° C. | 7 min | |
| 8° C. | old | |

### EXAMPLE 7

Gene Expression Analysis

The gene expression levels of the StGH1 and StGH2 genes were analysed in the transgenic potato lines with real-time PCR (ABI prism 7900HT, Applied Biosystems). With real-time PCR the change of gene expression can be analysed regarding RNA expression levels. For pHS1 and pHS2 transgenic lines, expression of both sense and antisense RNA of StGH1 and StGH2 was measured, while in pHS3 and pHS4 transgenic lines the change in StGH1 and StGH2 mRNA expression was analysed.

The target for pHS1 and pHS2 is to reduce transcript levels of StGH1 and StGH2 respectively while the target for pHS3 and pHS4 is to increase transcript levels of the respective genes.

RNA was isolated from microtubers of the transgenic potato lines and mother varieties using Invisorb Spin Plant-RNA mini kit (Invitek). A reverse transcription reaction was made with 250 ng total RNA in 25 μl total reaction volume using TaqMan reverse transcription reagents (Applied Biosystems). Separate and specific primers (see table 1) were designed and used for the reverse transcription reaction in order to be able to differentiate the endogenous expression from the antisense RNA expression of the respective genes.

TABLE 1

| | |
|---|---|
| StGH1 sense RNA | 5′-TGAAGACAGCACAAAACTGG-3′ |
| StGH1 antisense RNA | 5′-GTGAAAGTTTGAACGCACAC-3′ |
| StGH2 sense RNA | 5′-AGTGCCATAACATGCTTTCC-3′ |
| StGH2 antisense RNA | 5′-CACATTTCAGCTGTTGATGGA-3′ |

5 μl of the reverse transcription reaction was used in triplicate analyses together with specific sequence detection primers, TaqMan MGB probe (see table 2) and UMM mastermix (Applied Biosystems) and determined with real-time PCR according to the suppliers instructions.

TABLE 2

StGH1

| | |
|---|---|
| Forward Primer: | TCGAGTCGCCACGTAGAACTC |
| Reverse primer: | GAAATGCGTATGCGACTATGATG |
| TaqMan probe: | AGTCTCTCGGAGTTCC |
| StGH2 | |
| Forward primer: | GGTGCTGATCCTCCAGTTCTCT |
| Reverse primer: | GTCCCTGAAGCATAACCAAGGT |
| TaqMan probe: | TTCTGCACTACTTAGGCCT |

Down-regulation of the two genes resulted in a decrease in gene expression in transgenic lines compared to their mother varieties in the order of 50-95%.

Over-expression of the two genes resulted in a 2-10 times increase in gene expression in transgenic lines compared to their mother varieties.

TABLE 3
Gene expression analysis based on Real-Time PCR

| | | | | Times increase |
|---|---|---|---|---|
| | | | | or decrease in gene |
| | | | | expression compared |
| Line No. | Construct | Variety | Δ Ct s | to parental line |
| P01-041-84 | pHS1 | Producent | −1.14 | −1.3 |
| P02-325-1 | pHS1 | Producent | −2.03 | −4.1 |
| P02-325-9 | pHS1 | Producent | −1.47 | −2.2 |
| P02-325-11 | pHS1 | Producent | −1.25 | −1.6 |
| P02-325-15 | pHS1 | Producent | −2.52 | −6.3 |
| P02-325-25 | pHS1 | Producent | −2.0 | −4 |
| P02-325-27 | pHS1 | Producent | −1.64 | −2.7 |
| P02-325-33 | pHS1 | Producent | −1.59 | −2.5 |
| P02-325-34 | pHS1 | Producent | −1.52 | −2.3 |
| P02-325-63 | pHS1 | Producent | −1.53 | −2.3 |
| P02-300-37 | pHS2 | Prevalent | −1.27 | −1.6 |
| P02-300-66 | pHS2 | Prevalent | −1.04 | −1.1 |
| P02-300-71 | pHS2 | Prevalent | −1.13 | −1.3 |
| P02-300-73 | pHS2 | Prevalent | −1.1 | −1.2 |
| P02-300-80 | pHS2 | Prevalent | −2.12 | −4.5 |
| P02-300-127 | pHS2 | Prevalent | −1.67 | −2.8 |
| P02-300-140 | PHS2 | Prevalent | −3.96 | −15.7 |
| P02-303-31 | pHS2 | Prevalent | −1.16 | −1.4 |
| P02-303-64 | pHS2 | Prevalent | −1.15 | −1.3 |
| P02-305-54 | pHS2 | Prevalent | −1.33 | −1.8 |
| P02-320-24 | pHS2 | Prevalent | −1.03 | −1.1 |
| P02-307-4 | pHS3 | Desirée | 1.82 | 3.3 |
| P02-307-5 | pHS3 | Desirée | 2.68 | 7.2 |
| P02-307-12 | pHS3 | Desirée | 2.67 | 7.1 |
| P02-307-14 | pHS3 | Desirée | 1.83 | 3.3 |
| P02-307-15 | pHS3 | Desirée | 1.79 | 3.2 |
| P02-307-33 | pHS3 | Desirée | 3.21 | 10.3 |
| P02-307-43 | pHS3 | Desirée | 2.7 | 7.3 |
| P02-307-51 | pHS3 | Desirée | 2.73 | 7.5 |
| P02-307-80 | pHS3 | Desirée | 2.78 | 7.7 |
| P02-307-87 | pHS3 | Desirée | 1.02 | 1.1 |
| P02-307-148 | pHS3 | Desirée | 1.88 | 3.5 |
| P02-309-63 | pHS3 | AM99-2003 | 1.64 | 2.7 |
| P02-309-111 | pHS3 | AM99-2003 | 1.34 | 1.8 |
| P02-309-106 | pHS3 | AM99-2003 | 1.75 | 3.1 |
| P02-311-59 | pHS3 | AM99-2003 | 1.17 | 1.4 |
| P02-312-15 | pHS4 | AM99-2003 | 1.03 | 1.1 |
| P02-313-21 | pHS4 | AM99-2003 | 1.54 | 2.4 |
| P02-317-2 | pHS4 | AM99-2003 | 1.2 | 1.4 |

### EXAMPLE 8

Dry Matter Analysis

Dry matter has been analyzed on microtubers from transgenic lines transformed with pHS1, pHS2, pHS3 and pHS4 showing a down-regulation or over expression of the genes. Since starch normally contribute to more than 80% of the dry matter in potato tubers, an increase or decrease in starch content will affect also the dry matter content.

Two microtubers of each line were harvested when they had reached maturity. Dry matter was calculated for mature microtubers weighed before and after 72 hours drying at 60° C. For comparison microtubers from the varieties Dinamo, Desiree, Prevalent, Producent and P737 with starch contents between 13 and 28% (when grown in field) were used. The starch content of microtubers is not as high as starch content of field grown tubers. However dry matter content can readily be compared in microtubers and that value is well correlated to the determined starch content in field grown tubers. In table 4 the average dry matter for the different varieties, calculated on ten or more microtubers, is shown.

TABLE 4
Dry matter content of 5 varieties based on 10 or more microtubers

| | Starch content field | Dry matter |
|---|---|---|
| Variety | grown tubers | microtubers |
| AM99-2003 | 13% | 14.8 |
| Desirée | 16% | 16.1 |
| Producent | 22% | 19.2 |
| Prevalent | 22% | 19.7 |
| P737 | 28% | 21.6 |

One of each pHS1 and pHS2 with confirmed decrease in gene-expression have been analyzed for dry matter so far. Those two have a decrease in dry matter of 7 and 11% compared to their mother varieties.

For the pHS3 lines 8 of 9 of the confirmed over-expressed lines show an increase of up to 36% in dry matter. See table 5.

TABLE 5
Dry matter content on transgenic lines with confirmed
down-regulation or over-expression of the StGH1 and StGH2 genes

| | | | Dry matter in relation to parental |
|---|---|---|---|
| Line No. | Construct | Variety | line (%) |
| 41-84 | pHS1 | Producent | 89 |
| 300-127 | pHS2 | Prevalent | 93 |
| 300-140 | PHS2 | Prevalent | 96 |
| 307-4 | pHS3 | Desirée | 106 |
| 307-5 | pHS3 | Desirée | 117 |
| 307-15 | pHS3 | Desirée | 124 |
| 307-33 | pHS3 | Desirée | 116 |
| 307-57 | pHS3 | Desirée | 136 |
| 309-63 | pHS3 | AM99-2003 | 134 |
| 309-106 | pHS3 | AM99-2003 | 109 |
| 309-111 | pHS3 | AM99-2003 | 108 |

### EXAMPLE 9

Starch Content Analysis

For analysis of starch content a total starch assay procedure from Megazyme Interational Ireland Ltd., Bray, Co. Wicklow, Ireland (AOAC Method 996.1; MCC method 76.13; ICC standard method No. 168) was used according to the suppliers instructions.

Starch content was analysed on microtubers from all transgenic lines transformed with pHS1, pHS2, pHS3 and pHS4. The microtubers were harvested when they had reached maturity. Mature microtubers were ground and maltosaccharides and free glucose residues were washed away with ethanol. The microtuber starch was treated with DMSO to ensure the complete solubilisation of samples with high levels of resistant starch, as the high amylose clones.

Samples were analyzed with a standard spectrophotometric assay procedure. The transgenic lines were compared to potato varieties with known starch content ranging from 8% to 30%. The results give an indication on the change in starch content related to the genetic modification of the different transgenic lines.

### EXAMPLE 10

Greenhouse Trial

Harvest weight and dry matter content was measured on pHS3 and pHS4 transgenic lines and their mother varieties grown in the greenhouse. The harvest weight from 10 greenhouse grown pots was measured, see table 6.

TABLE 6
Greenhouse harvest weight of lines over-expressing
StGH1 (pHS3) or StGH2 (pHS4).

| | | | | Increase in harvest |
|---|---|---|---|---|
| | | Parental | Harvest | weight compared to |
| Line No. | Construct | line | weight (g) | parental line % |
| AM99-2003 | | | 1150 | |
| Desirée | | | 1500 | |
| P02-307-4 | pHS3 | Desirée | 1900 | 27 |
| P02-307-5 | pHS3 | Desirée | 2050 | 37 |
| P02-307-12 | pHS3 | Desirée | 2250 | 50 |
| P02-307-15 | pHS3 | Desirée | 1950 | 30 |
| P02-307-33 | pHS3 | Desirée | 1950 | 30 |
| P02-307-43 | pHS3 | Desirée | 2000 | 33 |
| P02-307-51 | pHS3 | Desirée | 1950 | 30 |
| P02-307-80 | pHS3 | Desirée | 1750 | 17 |
| P02-309-63 | pHS3 | AM99-2003 | 1550 | 35 |
| P02-309-114 | pHS3 | AM99-2003 | 1250 | 9 |
| P02-316-25 | pHS3 | AM99-2003 | 1450 | 26 |
| P02-316-111 | pHS3 | AM99-2003 | 1300 | 13 |
| P02-314-4 | pHS4 | Desirée | 1650 | 10 |
| P02-314-15 | pHS4 | Desirée | 1600 | 7 |
| P02-314-35 | pHS4 | Desirée | 1850 | 23 |
| P02-314-40 | pHS4 | Desirée | 1700 | 13 |
| P02-312-15 | pHS4 | AM99-2003 | 1350 | 17 |
| P02-313-21 | pHS4 | AM99-2003 | 1300 | 13 |
| P02-313-42 | pHS4 | AM99-2003 | 1650 | 43 |
| P02-317-2 | pHS4 | AM99-2003 | 1350 | 17 |

The harvest weight was increased up to 43% in the transgenic lines compared to their mother varieties. The results show that over-expression of StGH1 and StGH2 results in an increase in total harvest weight.

The lines grown in the greenhouse were analyzed for dry matter content. Slices of three tubers of each line were dried in a freeze dryer for 72 hours with weighing prior and after the drying. The dry matter results can be seen in table 7 and present a mean value of the three analyses.

TABLE 7
Analysis of dry matter content in lines over-expressing StGH1
(pHS3) or StGH2 (pHS4) grown in field trial.

| | | | | Increase in dry |
|---|---|---|---|---|
| | | | | matter in |
| | | | | comparison with |
| Line No. | Construct | Parental line | Mean value | parental line % |
| AM99-2003 | | | 19.15 | |
| Desiree | | | 19.25 | |
| P02-307-4 | pHS3 | Desiree | 21.29 | 11 |
| P02-307-5 | pHS3 | Desiree | 19.92 | 3 |
| P02-307-12 | pHS3 | Desiree | 22.46 | 17 |
| P02-307-14 | pHS3 | Desiree | 20.45 | 6 |
| P02-307-15 | pHS3 | Desiree | 22.12 | 15 |
| P02-307-33 | pHS3 | Desiree | 21.03 | 9 |
| P02-307-43 | pHS3 | Desiree | 19.68 | 2 |
| P02-307-80 | pHS3 | Desiree | 19.93 | 4 |
| P02-309-63 | pHS3 | AM99-2003 | 21.24 | 11 |
| P02-309-111 | pHS3 | AM99-2003 | 23.44 | 22 |
| P02-309-114 | pHS3 | AM99-2003 | 22.55 | 18 |
| P02-311-59 | pHS3 | AM99-2003 | 21.83 | 14 |
| P02-316-25 | pHS3 | AM99-2003 | 19.60 | 2 |
| P02-316-111 | pHS3 | AM99-2003 | 24.18 | 26 |
| P02-318-12 | pHS3 | AM99-2003 | 23.27 | 22 |
| P02-314-4 | pHS4 | Desiree | 19.77 | 3 |
| P02-313-21 | pHS4 | AM99-2003 | 21.08 | 10 |
| P02-313-42 | pHS4 | AM99-2003 | 20.61 | 8 |
| P02-317-2 | pHS4 | AM99-2003 | 20.76 | 8 |
| P02-317-15 | pHS4 | AM99-2003 | 19.45 | 2 |

Analyses of the transgenic lines over expressing the StGH1 or StGH2 genes show an increase in dry matter content compared to its respective parental line. As can be seen in table 7 the dry matter is increased up to 26% in lines over-expressing the StGH1 gene. The increase in dry matter is more pronounced when AM99-2003 is used as parental line. This is due to the fact that AM99-2003 is containing a significant amount of available sugars as a consequence of the high amylose trait (see FIG. 2). Also in lines over-expressing StGH2 the dry matter is increased. The increase is also in his case higher when AM99-2003 is used as parental line. Lines over-expressing StGH2 have an increase in dry matter of up to 10%.

### EXAMPLE 11

Field-Trial of Transgenic Potato Lines

Transgenic lines as described in examples 5 to 10 are tested in field trials for the determination of agronomic performance in relation to the parental line and other varieties used for starch production. Starch content, which is a main agronomic factor of importance for crops used for starch processing, can be measured by several different methods.

Under water weighing of tubers is performed on a scale in a tub of water. Starch content was determined according to standard procedure. 5 kg potato is used for the measurement and starch content is calculated according to the formula: Starch content in %=(density of potato−1.01506)/0.0046051. An increase in starch content is associated with an increase in the density of the sample. An increase of starch in the tubers is associated with an increased dry matter content, which can be measured by comparing the tissue fresh weight to tissue dry weight after extensive water elimination in an oven at 105° C. for 16 hours.

Starch content can also be measured by enzymatic methods as described under starch content analysis in example 9 and 12.

### EXAMPLE 12

Results in Field-Trial

Five lines over-expressing StGH1 or StGH2 were grown in the field as cuttings. The growth period was within June to September. After harvest the lines where analyzed for dry matter content, starch content and sugar content, see results in tables 8-10.

The dry matter content was analyzed by drying 15 g of mashed potatoes (produced in a blender) in a fanned heating oven for 16-18 hours at 105° C. The samples were cooled down to room temperature in an exicator before measurement.

The starch content from field grown tubers was analyzed according to an enzymatic method described in P. Aman et al. Methods in Carbohydrate Chemistry Vol. X. 1994, pp. 111-115 by using a thermostable α-amylase. Duplicate analysis was made on ground and dried samples of tubers diluted in ethanol (80%) and digested by thermostable α-amylase and amyloglucosidase. The amount of starch was determined by a glucose oxidase reaction.

The concentration of fructose, glucose and sucrose was determined using gas-liquid chromatography by methods described by Georg Fuchs et al., Swedish J. Agric. Res. 4:49-52, 1974, Quantitative determination of low-molecular carbohydrates in foods by gas-liquid chromatography.

TABLE 8
Analysis of dry matter content in lines over-expressing StGH1 or StGH2
grown in field-trials. Results presented are mean values of two analyses.

| | | parental | | Increase in |
|---|---|---|---|---|
| Line No. | Construct | line | Dry matter % | dry matter % |
| AM99-2003 | | | 20.7 | |
| Desiree | | | 21.8 | |
| P02-307-33 | pHS3 | Desiree | 22.6 | 4 |
| P02-307-80 | pHS3 | Desiree | 22.3 | 2 |
| P02-309-63 | pHS3 | AM99-2003 | 21.9 | 6 |
| P02-309-106 | pHS3 | AM99-2003 | 21.2 | 2 |
| P02-313-21 | pHS4 | AM99-2003 | 22.2 | 7 |

As can be seen in table 8 the field grown transgenic lines over-expressing StGH1 or StGH2 show an increase in dry matter of up to 7%. The lines also show an increase in starch content as can be seen in table 9. The highest increase can be seen for lines with AM99-2003 as parental line. This is due to the access of sugars available in the high amylose parental line (FIG. 2).

TABLE 9
Analysis of starch content determined by an enzymatic and a gravimetric
method. The lines over-expressing StGH1 (pHS3) or StGH2 (pHS4) were
grown in field-trials.

| | | | | Increase of |
|---|---|---|---|---|
| | | parental | | starch |
| Line No. | Construct | line | Starch content % | content % |
| AM99-2003 | | | 13.3 | |
| Desiree | | | 16.7 | |
| P02-307-33 | pHS3 | Desiree | 17.5 | 5 |
| P02-309-63 | pHS3 | AM99-2003 | 14.6 | 10 |
| P02-309-106 | pHS3 | AM99-2003 | 14.9 | 12 |
| P02-313-21 | pHS4 | AM99-2003 | 15.4 | 16 |

Furthermore the sugar concentrations were analyzed in lines with AM99-2003 as parental line. AM99-2003 contains a high fraction of available sugars due to the high amylose trait. In the lines over-expressing the StGH1 or the StGH2 gene the concentration of glucose has been reduced to ⅓ and sucrose has been reduced to ¾ of the amount analyzed in the parental line (see table 10). The lower sugar concentrations in the lines over-expressing the StGH1 or StGH2 gene show that more glucose and sucrose has been incorporated in the starch biosynthesis resulting in an increase in starch content in these transgenic lines.

TABLE 10
Analysis of fructose, sucrose and glucose content in lines over-expressing
StGH1 (pHS3) or StGH2 (pHS4) grown in field trial.

| | | | | | Decrease in | | Decrease in |
|---|---|---|---|---|---|---|---|
| | | parental | Fructose | Glucose | glucose | Sucrose | sucrose |
| Line No. | Construct | line | % of DM | % of DM | content % | % of DM | content % |
| AM99-2003 | | | 0.01 | 1.2 | | 3.25 | |
| P02-309-63 | pHS3 | AM99-2003 | 0.02 | 0.91 | −24 | 2.74 | −16 |
| P02-309-106 | pHS3 | AM99-2003 | 0.01 | 0.62 | −48 | 2.56 | −21 |
| P02-313-21 | pHS4 | AM99-2003 | 0.01 | 0.39 | −68 | 2.39 | −26 |

DM = dry matter.

### EXAMPLE 13

Microscopic Investigation of Lines Over-Expressing the StGH1 or the StGH2 Gene.

Field grown tubers of transgenic lines over-expressing the StGH1 or the StGH2 gene with AM99-2003 as parental line were investigated for starch granule morphology by staining starch with iodine (Lugol's solution (6.7 g/l KI+3.3 g/l I₂) and glycerol 1:1). A piece of a tuber was crushed and a few drops of iodine solution were added. The starch granule structure was analyzed under the microscope.

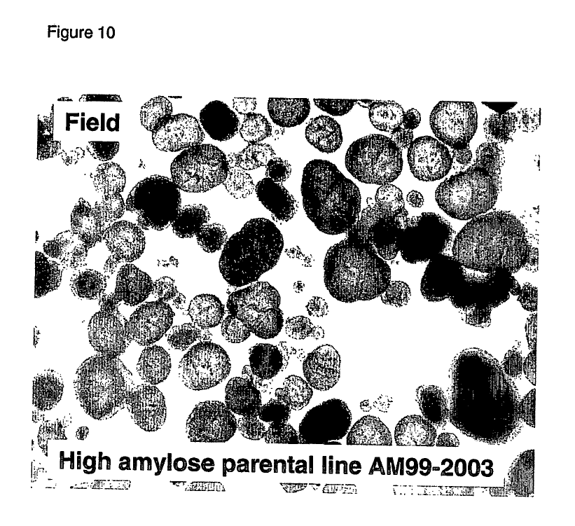

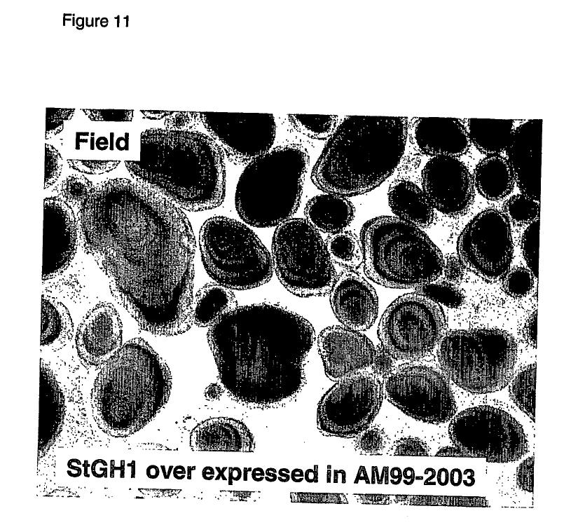

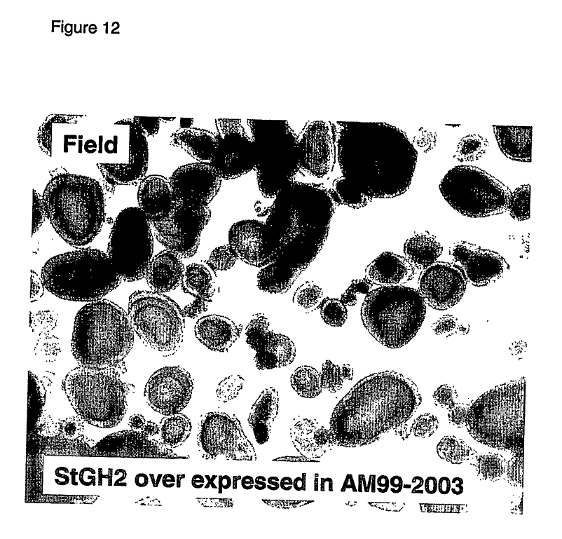

As can be seen in the FIG. 10, the starch granules are collapsed towards the interior of the granule in the high amylose parental variety AM99-2003. In contrast to this, the starch granules from the transgenic lines over-expressing the StGH1 or the StGH2 gene are—see FIGS. 11 and 12—larger and rounded in shape. This is due to the increased starch incorporation in the granules as a result of the over-expression of the StGH1 or the StGH2 gene.

### EXAMPLE 14

Combined Expression of Genes Related to the Starch Initiation

StGH1 and StGH2 can be combined in different ways. The genes can be combined on the same T-DNA or be located on separate T-DNAs. The genes can be used for co-transformation or be combined by crossing of transgenic lines.

### EXAMPLE 15

Combined Constructs for Inhibition of SBE1 and SBE2 and Over-Expression of StGH1 or StGH2

Constructs were made for production of high amylose lines containing high starch content. The constructs pHASHS2, pHASHS4, pHASHS5 and pHASHS6 were made for over-expression of StGH1 or StGH2. All pHASHS constructs also contain fragments of be1 and be2 for down-regulation of respective genes with the antisense technique or RNA interference technique (RNAi). The down-regulation of the SBE1 and SBE2 genes inhibits the amylopectin biosythesis and directs the starch biosythesis towards increased amylose production. All constructs are based on the binary vector pSUNA-HASmodb. The RNAi constructs are based on vector pHAS8b and the antisense constructs are based on vector pHAS4b.

Plants transferred with pHASHS2, pHASHS4, pHASHS5 or pHASHS6, yielded high-amylose lines. The starch content In the produced transgenic lines was higher than in high-amylose lines not over-expressing the StGH1 or StGH2 gene. The starch content was in the same range as can be seen for pHS3 and pHS4 lines described above.

### EXAMPLE 16

Combined Constructs for Inhibition of SBE1 and SBE2 and Over-Expression of StGH1 and StGH2

Constructs were made for production of high amylose lines containing high starch content. pHASHS7 and pHASHS8 were made for over-expression of StGH1 and StGH2 together in one plant. All pHASHS constructs also contain fragments of SBE1 and SBE2 for down-regulation of respective genes with the antisense technique or RNA interference technique (RNAi). The down-regulation of the SBE1 and SBE2 genes inhibits the amylopectin biosythesis and directs the starch biosythesis towards increased amylose production. All constructs are based on the binary vector pSUNA-HASmodb. The RNAi constructs are based on vector pHAS8b and the antisense constructs are based on vector pHAS4b.

Plants transformed with pHASHS7 and pHASHS8 yielded high-amylose lines. The starch content in the produced transgenic lines was higher than in high-amylose lines not over-expressing the StGH1 and StGH2 genes together.

### EXAMPLE 17

Vector Constructions

Construction of pSUNAHASmodb

A binary vector based on pSUN1 with a mutated AHAS gene as selection marker was constructed. The vector was used for further cloning of trait genes.

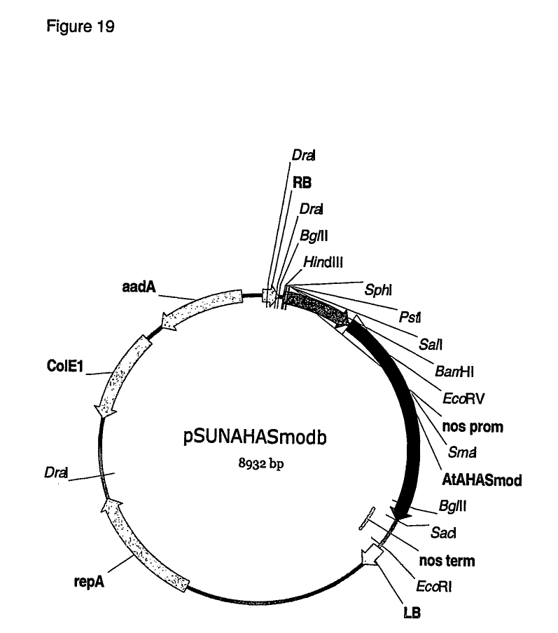

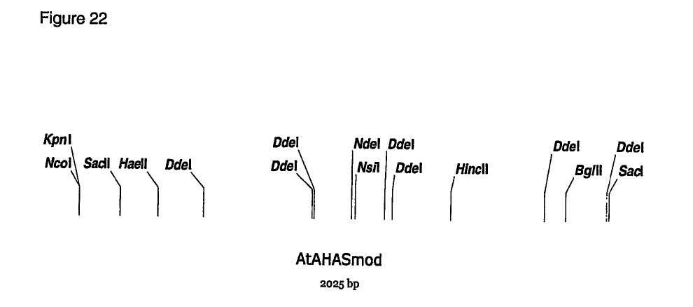

A 608 bp fragment containing the nos promoter was cut out from pGPTV-kan with Hind III and BglII and was ligated to pUC19 (Invitrogen) cut open with HindIII and BamHI. The nos terminator (275 bp) was cut out from pGPTV-kan with SstI and EcoRI and ligated to above between the SstI and EcoRI sites. The AHAS gene (S653N) described by Sathasivan et al (1991) was optimised by elimination of the restriction sites HindIII, EcoRV, BamHI, EcoRI and SstI by using QuikChange Multi Site directed Mutagenesis kit (Stratagene). Additional restriction sites, KpnI and SstI was added at the 3′ and 5′ ends of the gene. The gene was named AtAHASmod (FIG. 22, SEQ ID NO 16). AtAHASmod was cut with KpnI and SstI (ca 2019 bp) and ligated between the nos promoter and nos terminator at KpnI-SstI. The above complex was cut out from pUC19 with HindIII (blunt) and EcoRI (2900 bp) and ligated to pSUN1 at EcoRI and SmaI. The vector was named pSUNAHASmodb, see FIG. 19.

Construction of pHAS8b

An RNAi construct pHAS8b with a be1 and be2 fragment (SEQ ID NO 20) for down-regulation of the branching enzyme genes was constructed in the binary vector pSU-NAHASmodb (FIG. 19) based on pSUN1 with a mutated AHAS gene (SEQ ID NO 16) as selection marker. As spacer a fragment of the be2 promoter was used (SEQ ID NO 18). The vector was used for extended cloning with the StGH1 (SEQ ID NO 1) and StGH2 (SEQ ID NO 3) genes.

A 400 bp synthetically produced fragment of be2 (200 bp) and be1 (200 bp) in pBluescript named RNAi420be2be1 (SEQ ID NO 19) was opened with HindIII (blunt) and SalI (3331 bp). A 262 bp fragment of the be2 promoter (SEQ ID NO 18), for use as spacer, was digested with BglII (blunt) and SalI and ligated to the vector and named pMA17.

Again the 400 bp RNAi420be2be1 fragment was used and ligated in inverted direction to pMA17 opened with XhoI (blunt) and KpnI (3582 bp). The construct was named pMA18. pMA18 was digested with SpeI and KpnI (1120 bp) and the fragment was ligated between a gbss promoter and a nos terminator in pUC19 at XbaI and KpnI (3924 bp). The construct was named pMA19b. pMA19b was digested with PvulI and HindIII (2390 bp) and ligated to the binary vector pSUNAHASmodb (FIG. 19) between SphI (blunt) and HindIII (8932 bp). The construct was named pHAS8b, see FIG. 20.

Construction of pHAS4b

A vector pHAS4b with an antisense fragment for down-regulation of SBE1 and SBE2 was constructed in the binary vector pSUNAHASmodb (FIG. 19) based on pSUNi with a mutated AHAS gene (SEQ ID NO. 16) as selection marker. The vector was used for extended cloning of the StGH1 and the StGH2 genes.

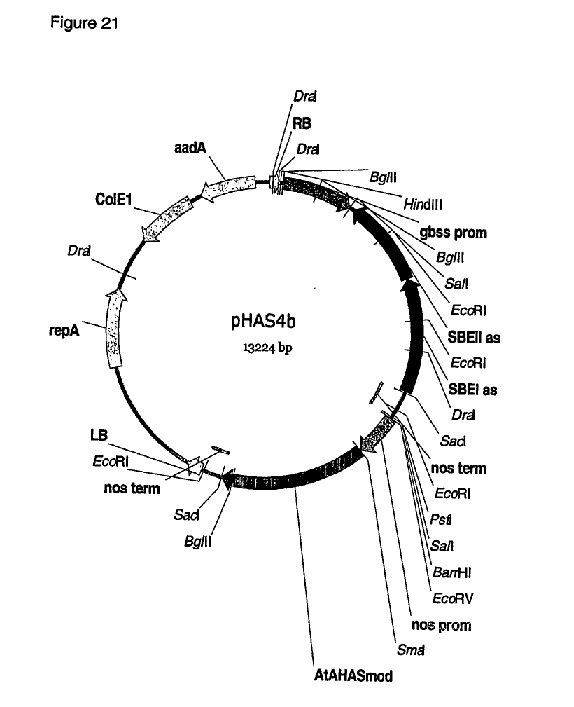

The antisense fragment of be1 and be2 together with gbss promoter and nos terminator was cut out of pHAbe12A (FIG. 9, SEQ ID NO 15) with BsrBI and HindIII (4299 bp) and ligated to pSUNAHASmodb (FIG. 19) digested with SphI (blunt) and HindIII (8932 bp). The construct was named pHAS4b, see FIG. 21.

Construction of pHASHS5

The StGH1 gene, the gbss promoter and the nos terminator was digested from pHS3 (FIG. 6) and cloned into the RNAi construct pHAS8b (FIG. 20) containing fragment of be1 and be2.

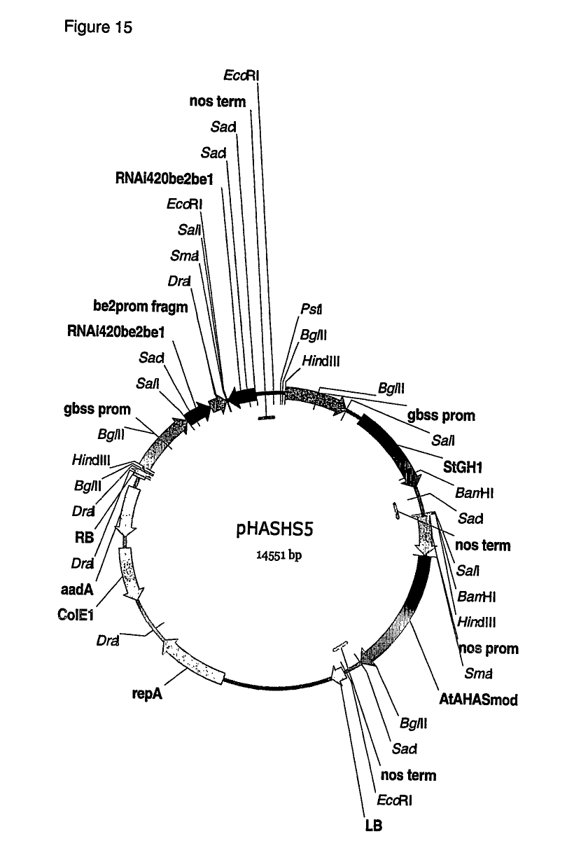

The StGH1 gene, gbss promoter and nos terminator was cut out from pHS3 with DraI and EcoRV (3160 bp). The fragment was ligated to pHAS8b opened up with EcoRV (11403 bp). The construct was named pHASHS5, see FIG. 15.

Construction of pHASHS6

The StGH2 gene, the gbss promoter and the nos terminator was digested from pHS4 (FIG. 7) and cloned into the RNAi construct pHAS8b (FIG. 20) containing fragments of be1 and be2.

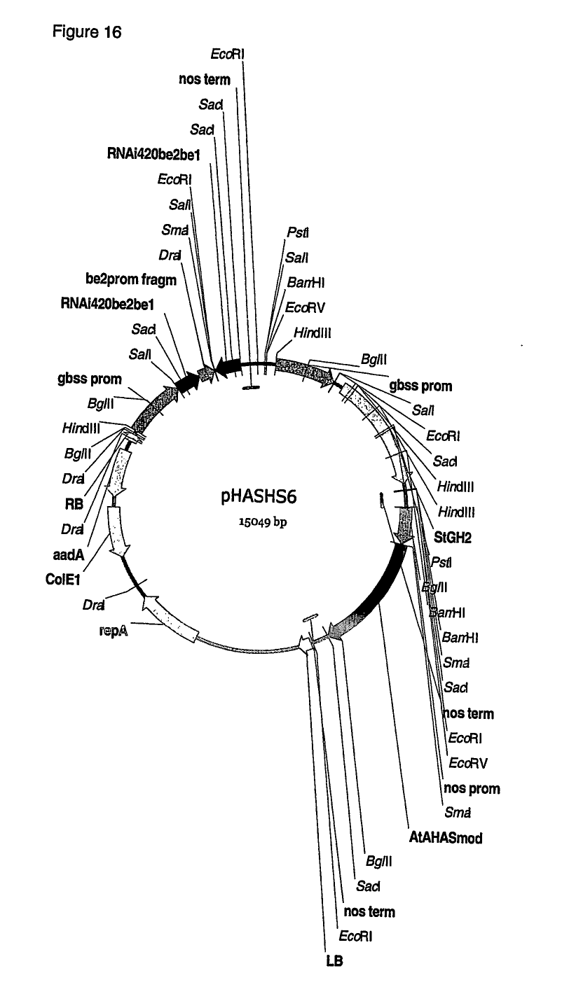

pHS4 was digested with SpeI and EcoRI. Two fragments were collected for further cloning, a 2486 bp EcoRI-EcoRI fragment and a 1169 bp SpeI-EcoRI fragment. pBluescript was digested with SpeI and EcoRI. The digested pBluescript was ligated with the 1169 bp (SpeI-EcoRI) fragment. The construct was named pMA15. pMA15 was digested with EcoRI (4127 bp) and ligated to the 2486 bp EcoRI-EcoRI fragment from pHS4.The construct was named pMA16. A 3689 bp fragment was cut out rom PMA16 digested with EcoRV and ligated to pHAS8b opened with EvoRV (11403 bp). The construct was named pHASHS6, see FIG. 16.

Construction of pHASHS2

The StGH1 gene, the gbss promoter and the nos terminator was digested from pHS3 (FIG. 6) and cloned into pHAS4b (FIG. 21) containing an antisense fragment of be1 and be2.

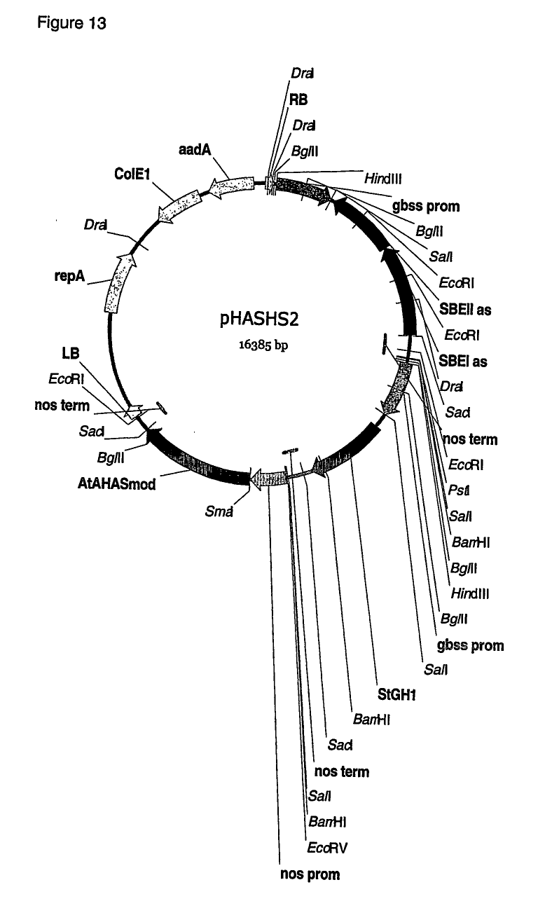

pHS3 was digested with DraI and EcoRV (3160 bp) and ligated to pHAS4b opened up with EcoRV (13224 bp). The construct was named pHASHS2, see FIG. 13.

Construction of pHASHS4

The StGH2 gene, the gbss promoter and the nos terminator was digested from pHS4 (FIG. 7) and cloned into pHAS4b (FIG. 21) containing an antisense fragment of be1 and be2.

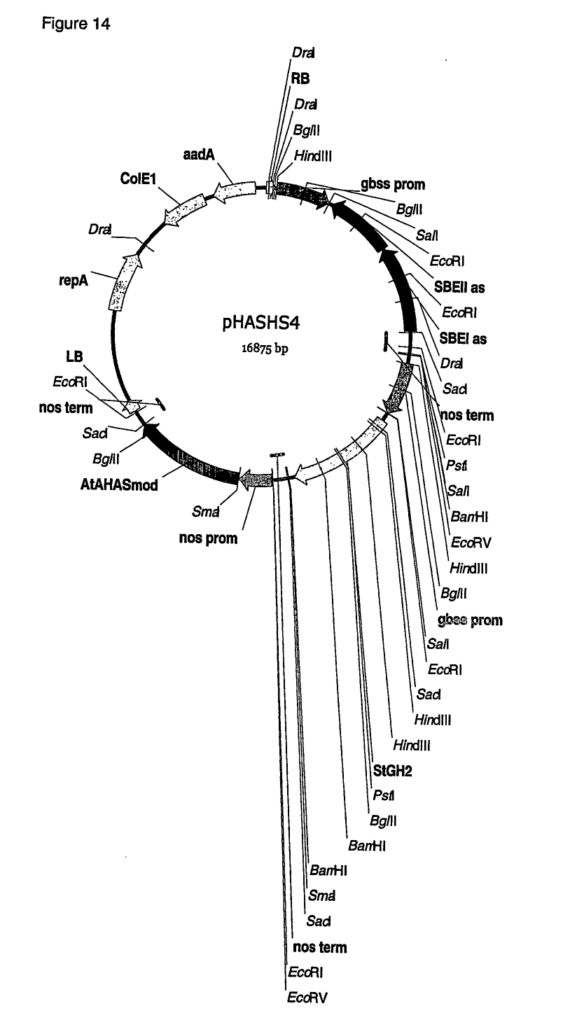

A 3689 bp fragment was digested from pMA16 with EcoRV (6613 bp) (for pMA16 see construction strategy of pHASHS3). pHAS4b was digested with EvoRV (13224 bp) and ligated with the above fragment. The construct was named pHASHS4, see FIG. 14.

Construction of pHASHS7

pHASHS7 was designed to contain antisense fragments of be1 and be2 for inhibition of respective gene together with the two amylose biosynthesis enhancing genes StGH1 and StGH2.

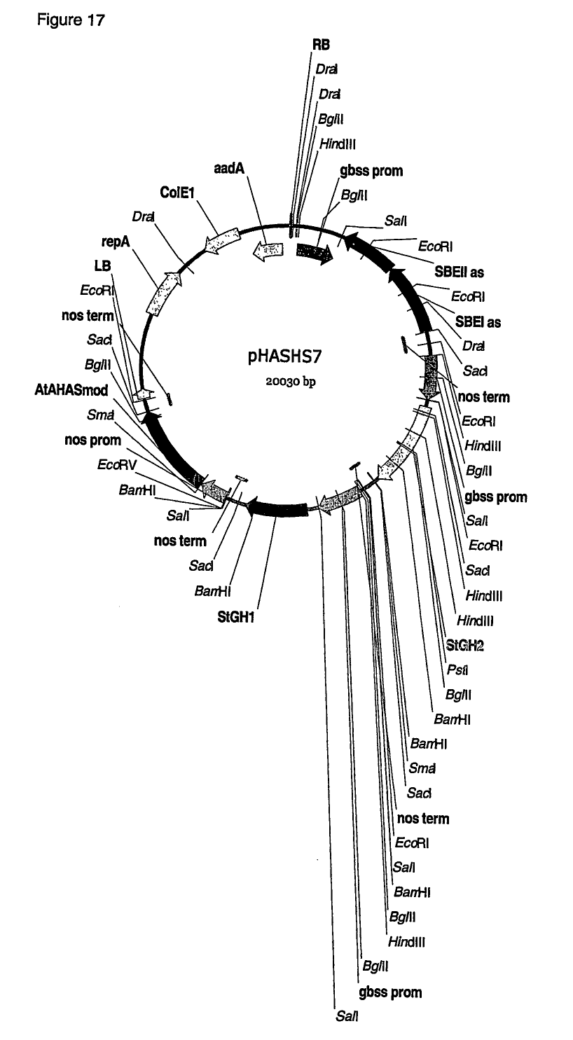

pMA16 (for pMA16 see construction strategy of pHASHS3) was digested with EcoRV. The resulting 3649 bp fragment was ligated to pHASHS2 (FIG. 5) opened with PstI (blunted). The construct was named pHASHS7, see FIG. 17.

Construction of pHASHS8

pHASHS8 was designed to contain a fragment of be1 and be2 for inhibition of respective gene using RNAi (SEQ ID NO 19 to 22) and linked by a spacer (SEQ ID NO 18 or 23) together with two amylose biosynthesis enhancing genes StGH1 and StGH2.

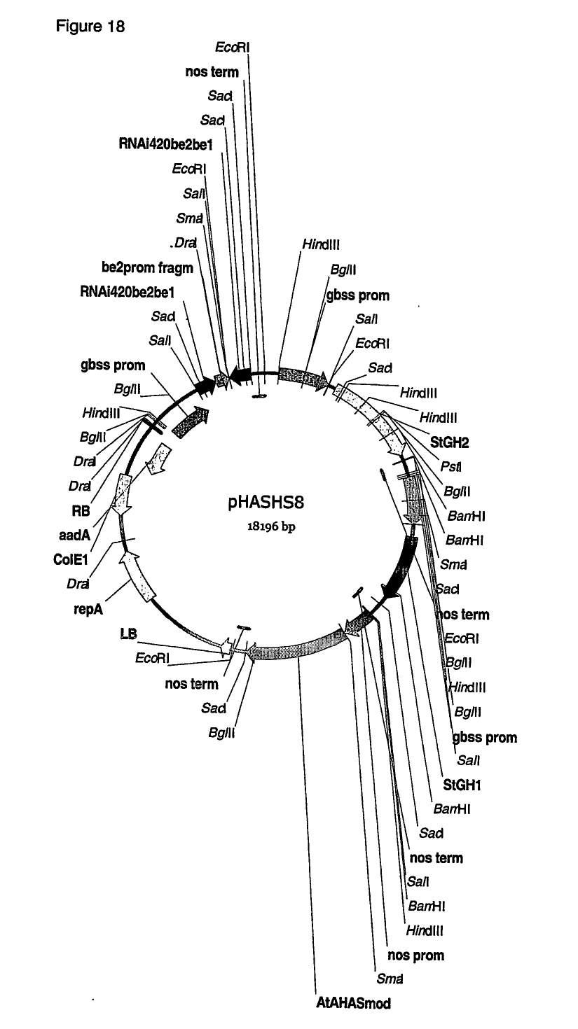

pMA16 (for pMA16 see construction strategy of pHASHS3) was digested with EcoRV and ligated to pHASHS5 (FIG. 15) opened with PstI and blunted. The construct was named pHASHS8, see FIG. 18.

### EXAMPLE 18

Increased Solids and Improved Processing Quality of Potatoes

In another aspect the invention may be used to increase the solids content of potato varieties that are used for processed potato products or as table potato varieties. The potato genotypes are transformed with gene constructs as described above for the over-expression of a gene coding for a starch biosynthesis enhancing protein. This starch biosynthesis enhancing protein may be derived from genes described above or other plant genes containing the same functional domains.

Over-expression of the StGH1 and/or the StGH2 gene in potato plants results in an increase in solids as can be seen in table 7 and 8.

1. A method of increasing the production of starch in plants comprising culturing a plant with enhanced expression or activity of at least one starch biosynthesis enhancing protein.

2. The method of claim 1, wherein said starch has a high amylose content.

3. The method as claimed in claim 1, wherein production of amylose is increased.

4. The method as claimed in claim 1, wherein said method comprises over-expression of a starch biosynthesis enhancing protein.

5. The method as claimed in claim 4, wherein said protein comprises the SEQ ID NO: 2 or 4 or a protein derived from this sequence by substitution, insertion or deletion of amino acids and which has at least 50% identity at the amino acid level with SEQ ID NO: 2 or 4.

6. The method as claimed in claim 1, wherein the starch biosynthesis enhancing protein is encoded by a nucleic acid sequence selected from the group consisting of:
a) a nucleic acid sequence comprising a nucleotide sequence which is at least 60% identical to the nucleic acid sequence of SEQ ID NO: 1 or 3;
b) a nucleic acid sequence comprising a fragment of at least 30 nucleotides of a nucleic acid sequence comprising the nucleotide sequence of SEQ ID NO: 1 or 3;
c) a nucleic acid sequence which encodes a polypeptide comprising an amino acid sequence at least about 60% identical to the amino acid sequence of SEQ ID NO:2 or 4 and
d) a nucleic acid sequence which encodes a fragment of a polypeptide comprising the amino acid sequence of SEQ ID NO: 2 or 4 or wherein the fragment comprises at least 10 contiguous amino acid residues of the amino acid sequence of SEQ ID NO:2 or 4,

7. The method as claimed in claim 1, wherein the starch biosynthesis enhancing protein is encoded by a nucleic acid sequence comprising the nucleotide sequence set forth as SEQ ID NO:1 or SEQ ID NO:3.

8. The method as claimed in claim 1, wherein deficiency or decreased activity is achieved by a method selected from the group consisting of:
a) knock-out of the gene encoding said protein;
b) mutagenesis of the gene encoding said protein, wherein said mutation can be induced in the coding, non-coding, or regulatory regions of said gene;
c) expression of an anti-sense RNA, wherein said anti-sense RNA is complementary to at least part of the RNA encoding said protein;

9. A method of producing amylose type starch by culturing a plant which over-expresses SEQ ID NO: 1 or 3 or has increased starch biosynthesis enhancing activity under conditions such that the plant produces an increased amount of amylose type starch.

10. The method of claim 1, wherein said plant belongs to the genus Solanum.

11. The method of claim 10, wherein said plant is Solanum tuberosum.

12. A nucleic acid sequence SEQ ID NO:1 encoding a starch biosynthesis enhancing protein.

13. A nucleic acid sequence SEQ ID NO:3 encoding a starch biosynthesis enhancing protein.

14. An amino acid sequence SEQ ID NO:2 having starch biosynthesis enhancing activity.

15. An amino acid sequence SEQ ID NO:4 having starch biosynthesis enhancing acitivity.

16. A transgenic expression cassette comprising in combination with a regulatory sequence a nucleic acid sequence selected from the group consisting of:
a) a nucleic acid sequence comprising a nucleotide sequence which is at least 60% identical to the nucleotide sequence of SEQ ID NO:1 or SEQ ID NO:3,
b) a nucleic acid sequence comprising a fragment of at least 30 nucleotides of a nucleic acid sequence comprising the nucleotide sequence of SEQ ID NO:1 or SEQ ID NO:3,
c) a nucleic acid sequence which encodes a polypeptide comprising an amino acid sequence at least about 60% identical to the amino acid sequence of SEQ ID NO:2 or SEQ ID NO:4, or
d) a nucleic acid sequence which encodes a fragment of a polypeptide comprising the amino acid sequence of SEQ ID NO:2 or SEQ ID NO:4 wherein the fragment comprises at least 10 contiguous amino acid residues of the amino acid sequence of SEQ ID NO:2 or SEQ ID NO:4
wherein said regulatory sequence is capable of mediating expression of said nucleic acid sequence in a plant.

17. A transgenic expression cassette of claim 16, wherein said regulatory sequence is a promoter sequence heterologous with regard to said nucleic acid sequence.

18. A transgenic expression cassette of claim 16, wherein said regulatory sequence is a tuber specific promoter sequence.

19. A transgenic expression cassette of claim 16, wherein said nucleic acid sequence is arranged in antisense or sense orientation with regard to said promoter sequence.

20. A transgenic expression cassette of claim 16, wherein said nucleic acid sequence encodes a polypeptide comprising the amino acid sequence set forth in SEQ ID NO:2 or SEQ ID NO:4.

21. A transgenic expression cassette of claim 16, wherein said nucleic acid molecule comprises the nucleotide sequence set forth in SEQ ID NO:1 or SEQ ID NO:3.

22. A transgenic expression cassette of claim 16, wherein said nucleic acid sequence encodes a naturally occurring variant of a polypeptide comprising the amino acid sequence set forth in SEQ ID NO:2 or SEQ ID NO:4.

23. A transgenic host cell transformed with an expression cassette of claim 16.

24. A transgenic host cell of claim 23, wherein said host cell belongs to the genus Solanum.

25. A transgenic plant comprising an expression cassette of claim 16.

26. A transgenic potato plant comprising an expression cassette of claim 16.

27. A transgenic potato plant, plant part, seed or tuber comprising an expression cassette of claim 16.
